(19) United States
(12) Patent Application Publication
(10) Pub. No.: US 2012/0148539 A1
(43) Pub. Date: Jun. 14, 2012

(54) Methods and Compositions for Tissue Engineering

(76) Inventor: Moulay Hicham Alaoui-Ismaili, Hopkinton, MA (US)

(21) Appl. No.: 13/256,856

(22) PCT Filed: Feb. 19, 2010
(86) PCT No.: PCT/US2010/024726

§ 371 Date: Sep. 15, 2011

Related U.S. Application Data
(60) Provisional application No. 61/162,821, filed on Mar. 24, 2009.

(51) Int. Cl. A61K 38/18; C12N 5/0775; A61P 19/00; A61K 35/32

(52) U.S. Cl. 424/93.7; 514/8.8; 435/377

(57) ABSTRACT

Disclosed herein are methods and compositions for promoting endochondral bone formation. The invention includes methods of promoting endochondral bone formation by down-regulating the expression of the DIO2 gene or the activity of the deiodinase protein. The invention also includes methods of up-regulating the activity of the FGFR3, ADAMTS9, HEY1, HAS3, and/or MFI2 genes and/or the activity of the expression products of those genes to promote endochondral bone formation. The invention also includes compositions of BMP-7 and agonists of FGFR3, ADAMTS9, HEY1, HAS3, and/or MFI2 proteins. Compositions can further include mesenchymal stem cells.

### CROSS REFERENCE TO RELATED APPLICATIONS

This application claims priority to and the benefit of U.S. Provisional Patent Application No. 61/162,821, the contents of which are incorporated by reference herein.

### FIELD OF THE INVENTION

The application is directed to methods and compositions useful in regulating the formation of various tissues, including bone and cartilage.

### BACKGROUND

Mesenchymal stem cells (MSC) are capable of differentiating into a variety of cell types, including osteoblasts, chondrocytes and adipocytes. Differentiation of MSC into osteoblasts is requisite to embryonic skeletal formation, homeostatic skeletal remodeling and fracture repair. Osteoblastogenesis encompasses numerous morphological and molecular changes through which MSCs acquire their ability to deposit the mineralized extracellular matrix (ECM) characteristic of bone tissue (Ducy et al., (2000), Science, 289:1501-4. This transformation is a multi-step process that is highly dependent on environmental signals and tightly regulated at multiple junctures.

Bone Morphogenetic Proteins (BMPs) are members of the Transforming Growth Factor-β (TGF-β) superfamily of growth factors, and are well established physiological regulators of osteoblastic differentiation. Several BMPs display osteoinductive activity, and two of these, BMP-7 (osteogenic protein-1, OP-1) and BMP-2, are currently used clinically to induce new bone formation in spine fusions and long bone non-union fractures (Gautschi et al., (2007), ANZ J. Surg. 77:626-31). BMP signaling is mediated by tetramers of serine/threonine kinase trans-membrane receptors, consisting of two type I and two type II receptors (Sebald et al., (2004), 385:697-710). BMP ligand/receptor binding leads to phosphorylation of type I receptors and activation of intracellular signaling proteins including the receptor-regulated Smads (R-Smads). R-Smads then form heteromeric complexes with the common mediator Smad, Smad-4, and translocate to the nucleus to induce transcription of BMP responsive genes.

Primary human Mesenchymal Stem Cells (hMSCs) represent an attractive model for studying BMP osteogenic bioactivity, as they are believed to be the primary cell type responsive to BMP signaling during bone formation in vivo. Nonetheless, the current understanding of molecular events induced by a single BMP during osteoblastic differentiation of hMSC is incomplete. In particular, such an understanding is critical to identification of materials and methods suitable for tissue engineering.

### SUMMARY OF INVENTION

The present invention is based on the discovery that an exemplary BMP, BMP-7, can influence discrete components of a cell differentiation pathway as well as the formation of differentiated tissue. As presented herein, BMP-7 can uniquely and profoundly influence aspects of cellular differentiation such that its utility as a targeted therapeutic and tissue engineering tool is evident to the skilled practitioner.

In one aspect, the present invention is directed to a method of promoting endochondral bone formation comprising the step of inhibiting or down-regulating the activity of deiodinase and/or the DIO2 gene. In another aspect, the present invention is directed to a method of promoting endochondral bone formation comprising the step of inducing or up-regulating the activity of the gene, or its expression product, selected from the group consisting of: FGFR3, ADAMTS9, HEY1, HAS3 and MFI2.

In yet another aspect, the present invention is directed to a method of promoting endochondral bone formation comprising the steps of (a) administering an effective amount of an agent which reduces or blocks the activity of deiodinase and/or the DIO2 gene in combination with an effective amount of BMP-7 and/or BMP-7 mimetic and/or BMP-7 agonist, wherein the administering step induces osteoblastogenesis but not mineralization of osteoblasts resulting therefrom; (b) allowing accumulation of non-mineralized osteoblasts; and, (c) reversing or neutralizing the agent's block of deiodinase and/or the DIO2 gene, wherein accumulated osteoblasts are mineralized and endochondral bone formation occurs. In a preferred embodiment, step (c) is accomplished upon metabolic depletion or exhaustion of the agent over time. In another preferred embodiment, step (c) is accomplished by providing: T3; an agonist of deiodinase; and/or an agonist of the DIO2 gene. In yet another preferred embodiment, the effective amount of BMP-7 is endogenous BMP-7. In still another preferred embodiment, the method is accomplished using an effective amount of an agent which reduces or blocks the activity of Noggin and/or the Noggin gene.

In another aspect of the present invention, the invention is directed to a pharmaceutical composition comprising BMP-7; and, an agent which reduces or blocks the activity of deiodinase and/or the DIO2 gene. In one embodiment, the agent is an inhibitor of deiodinase selected from the group consisting of: iopanoic acid, structural or functional analogs of iopanoic acid, naturally occurring chemical inhibitors, and non-naturally-occurring chemical inhibitors. In another embodiment, the agent is selected from the group consisting of: siRNA, structural or functional analogs of siRNA, antisense RNA, naturally occurring non-chemical inhibitors, and non-naturally-occurring non-chemical inhibitors.

In another aspect, the invention is directed to a pharmaceutical composition comprising BMP-7; and, an agent which reduces or blocks the activity of a T3 molecule. In yet another aspect, the invention is directed to a pharmaceutical composition comprising BMP-7; and, a T4 analog which prevents the formation of T3.

In another aspect, the invention contemplates a composition comprising stem cells; BMP-7 or mimetic or agonist thereof; and, an antagonist of deiodinase and/or the DIO2 gene and/or T3. In certain preferred embodiments, the composition further comprises an antagonist of Noggin and/or the Noggin gene.

In yet another aspect, the present invention is directed to a cell culture of continuously proliferating non-mineralized osteoblasts derived from human mesenchymal stem cells.

In still another aspect, the invention is directed to a method of modulating chondrogenesis comprising the step of providing BMP-7 or mimetic or agonist thereof in an amount effective to down-regulate GDF-5-mediated chondrogenesis, wherein osteoblastogenesis follows GDF-5 down-regulation.

In still another aspect, the invention contemplates a method of modulating endochondral bone formation comprising the step of providing BMP-7 or a mimetic or agonist thereof in an amount effective to down-regulate CHI3L1 gene activity, wherein bone deposition follows CHI3L1 down-regulation.

In yet another aspect, the present invention is directed to a method of tissue engineering comprising the step of providing BMP-7, BMP-7 mimetic or BMP-7 agonist in an amount effective transiently to attenuate stem cell cell-cycle events comprising attenuation of stem cell proliferation during early blastogenesis; or providing BMP-7 or agonist thereof in an amount effective continuously to down-regulate osteoclastic events comprising modulation of chemokine- or cytokine-induced osteoclastogenesis.

In another aspect, the invention contemplates a method of preparing non-mineralized differentiated osteoblasts comprising the step of contacting human mesenchymal stem cells with an effective amount of BMP-7 and an effective amount of an agent which reduces or blocks the activity of deiodinase and/or the DIO2 gene, wherein the cells undergo osteoblastogenesis to form differentiated osteoblasts which are non-mineralized.

In a related aspect, the invention contemplates a method of preparing non-mineralized differentiated osteoblasts comprising the step of contacting human mesenchymal stem cells with an effective amount of BMP-7 and an effective amount of an agent which reduces or blocks the activity of a T3 molecule, wherein the cells undergo osteoblastogenesis to form differentiated osteoblasts which are non-mineralized.

In a related aspect, the invention further contemplates a method of preparing non-mineralized differentiated osteoblasts comprising the step of contacting human mesenchymal stem cells with an effective amount of BMP-7 and an effective amount of a T4 analog which prevents the formation of T3, wherein the cells undergo osteoblastogenesis to form differentiated osteoblasts which are non-mineralized.

Still another aspect of the invention is directed to a method of promoting osteoblastogenesis of non-mineralized osteoblasts comprising the step of contacting human mesenchymal stem cells with an effective amount of BMP-7 and an effective amount of an agent which reduces or blocks the activity of deiodinase and/or the DIO2 gene, wherein the cells undergo osteoblastogenesis to form differentiated osteoblasts which are non-mineralized.

Still another aspect of the present invention is directed to a method of arresting osteoblastic differentiation of human mesenchymal stem cells comprising the step of contacting human mesenchymal stem cells with an effective amount of BMP-7 and an effective amount of an agent which reduces or blocks the activity of deiodinase and/or the DIO2 gene, wherein osteoblastic differentiation is arrested such that the cells undergo osteoblastogenesis but not mineralization.

One further aspect contemplates a method of continuous cultivation of partially differentiated human mesenchymal stem cells comprising the step of contacting human mesenchymal stem cells with an effective amount of BMP-7 and an effective amount of an agent which reduces or blocks the activity of deiodinase and/or the DIO2 gene, wherein the cells undergo osteoblastogenesis to form partially differentiated cells.

### BRIEF DESCRIPTION OF THE FIGURES

FIG. 1A is a line graph demonstrating AP activity in BMP-7 treated hMSC over a range of doses from 4 ug/ml to 16 ng/ml.

FIG. 1B shows the results of mineralization assays with hMSCs in response BMP-7 doses of 100-500 ng/mL or an ODM control after staining of cultures with Alizarin red.

FIG. 1C shows an Affymetrix HG-U133 Plus 2.0 Array analysis of gene expression of genes over the entire human genome in primary hMSC treated with BMP-7.

FIGS. 2A-F are line graphs demonstrating the fold change in gene expression of FGFR3 (A), DIO2 (B), HEY1 (C), ADAMTS9 (D), HAS3 (E), and MFI2 (F) genes over time for hMSCs treated with ODM alone or ODM supplemented with 40 or 400 ng/ml BMP-7.

FIG. 3A shows the results of BMP-7 mediated osteoblastic mineralization after treatment with siRNA to inhibit expression of various target genes (1) FGFR3, (2) DIO2, (3) HEY1, (4), ADAMTS9, (5) HAS3, and (6) MFI2. Values shown represent the mean±S.D. of triplicate measurements from one representative experiment. Percent inhibition of each targeted siRNA relative to the negative control siRNA from the same time point is shown.

FIG. 3B shows well plates of hMSCs stained with Alizarin red after 12 days of BMP-7 treatment and treatment with various siRNA targeted to expression of MFI2, HEY1, HAS3, FGFR3, and DIO2 to assess mineralization.

FIG. 3C shows well plates of hMSC nucleofected with siRNA targeting DIO2 stained with Alizarin red after 9 days of nucleofection. The cells show advanced mineralization prior to detachment.

FIG. 4A shows the cell cycle gene expression of CCNE2, ANLN, BRCA1 and CDC2 mRNA as regulated by BMP-7 and as quantified by RT-QPCR and normalized to GAPDH. Values shown represent the mean±S.D. of triplicate measurements and are expressed as percent change in gene expression in BMP-7 treated cells relative to the ODM control at each time point. Time points at which BMP-7 treatments are statistically different (p<0.05) from the control for all genes are indicated with an asterisk (*).

FIG. 4B is a bar graph showing analysis of hMSC treated with BMP-7 as analyzed by flow cytometry. Values shown represent the mean±S.D. of triplicate measurements. Data are expressed as the percent change in the number cells at each stage of the cell cycle in BMP-7 treated cells relative to cells treated with ODM alone. Bars representing significant differences between BMP-7 versus control treatments are marked with an asterisk (*), with p-values provided in text.

FIG. 4C exemplifies the number of hMSC over time after culturing in ODM alone or ODM supplemented with 500, 200 or 50 ng/ml BMP-7. Values shown represent the mean±S.D. of triplicate treatment wells. Data are expressed as the percent change in the number cells in BMP-7 treated cells relative to cells treated with ODM alone. Time points where significant differences between BMP-7 versus control treatments were observed are marked with an asterisk (*).

FIGS. 5A-F are line graphs showing how BMP-7 down-regulates cytokine expression in primary hMSC, in particular, hIL-6 (A), hIL-8 (B), hMCP-1 (C), hIFN-alpha (D), hHGF (E), and hVEGF (F) when cultured in ODM alone or ODM supplemented with 50 or 500 ng/ml BMP-7. Values shown represent the mean±S.D. of triplicate measurements and are expressed in pg/ml. BMP-7 treatments that are significantly different (p<0.05) from the control are indicated by dotted lines.

FIG. 6 is a line graph showing fold change in expression of CHI3L1 over time in primary hMSC cultured in ODM alone or ODM supplemented with 40 or 400 ng/ml BMP-7. BMP-7 down-regulates cartilage glycoprotein-39. Expression of CHI3L1 mRNA was quantified by RT-QPCR and normalized to GAPDH. Values shown represent the mean±S.D. of triplicate measurements and are expressed relative to treatment with ODM alone at day one. BMP-7 treatments that are significantly different (p<0.05) from the control are indicated by dotted lines.

FIGS. 7A-B are line graphs showing the fold change in expression of BMP-2 (A) and GDF-5 (B) over time in primary hMSC cultured in ODM alone or ODM supplemented with 40 or 400 ng/ml BMP-7. Values shown represent the mean±S.D. of triplicate measurements and are expressed relative to treatment with ODM alone at day one. BMP-7 treatments that are significantly different (p<0.05) from the control are indicated by dotted lines.

FIGS. 8A-D demonstrate that endogenous BMP-2 expression is not required for BMP-7 induced osteoblastic differentiation. Figure A is a bar graph showing BMP-2 expression over time in primary hMSC nucleoporated with a total of 4 μg siRNA targeting BMP-2 or positive or negative control siRNAs. Values shown represent the mean±S.D. of triplicate measurements from one representative experiment. Percent inhibition of each targeted siRNA relative to the negative control siRNA from the same time point is shown.

FIG. 8B shows control cells and cells treated with BMP-7 as stained with Alizarin red after 12 days of BMP-7 treatment to assess mineralization.

FIG. 8C shows is a bar graph showing fold change in calcium content assessed in hMSC after 10 days of BMP-7 treatment. Values shown represent the mean±S.D. of triplicate treatment wells from one representative experiment.

FIG. 8D is a bar graph showing DLX5 and Noggin gene expression after two days (Panels 1 & 2), ID1 (Panel 3) and SP7 (Panel 4) gene expression after four days, and PTHR1 (Panel 5) and IBSP (Panel 6) gene expression after eight days in hMSC cultured with BMP-7. Values shown represent the mean±S.D. of triplicate treatment wells and are expressed relative to BMP-7 treated cells nucleoporated with positive control siRNA.

FIGS. 9A-D are line graphs showing the fold change in expression of NOG (A), BAMBI (B), GREM1 (C), and GREM2 (D) over time in hMSC cultured in ODM alone or ODM supplemented with 40 or 400 ng/ml BMP-7. Values shown represent the mean±S.D. of triplicate measurements and are expressed relative to treatment with ODM alone at day one or to the earliest time point of detection. BMP-7 treatments that are significantly different (p<0.05) from the control are indicated by dotted lines.

### DETAILED DESCRIPTION OF THE INVENTION

The present invention is based on the heretofore undiscovered observation that osteoblastic differentiation is associated with regulation of several genes and pathways not previously associated with osteoblastic differentiation. In particular, the invention is based on the observation that these genes and pathways are influenced by, for example, BMP-7.

Based on Affymetrix gene profiling of BMP-7 mediated gene expression during early osteoblastic differentiation of primary hMSC (see Example 2 below), Applicants discovered that six genes—FGFR3, ADAMTS9, HEY1, HAS3, MFI2, and DIO2—with heretofore undefined roles in osteoblastic differentiation were subject to striking up-regulation. A functional screen using siRNA (see Example 4 below) suggested that two of these genes, MFI2 and HEY1, were essential to mineralization in differentiating hMSC.

MFI2 is an iron-binding cell-surface glycoprotein that shares sequence similarity with members of the transferrin superfamily. Proteins of this superfamily contribute to iron sequestration in osteoblast cells (Spanner et al., (1995), Bone, 17:161-5), and have been shown to stimulate osteoblastogenesis and inhibit osteoclastogenesis in vitro, and enhance bone formation in vivo (Cornish et al., (2004), Endocrinology, 145:4366-74; Cornish et al., (2006), Biochem. Cell Biol., 84:297-302).

HEY1 is a nuclear protein belonging to the hairy and enhancer of split-related family of basic helix-loop-helix-type transcriptional repressors. HEY1 is highly induced by BMP-9 in murine C3H10T1/2 cells and required for osteogenic differentiation (Sharff et al., (2008), J. Biol. Chem, 284:649-59), indicating a role for HEY1 in BMP-driven osteoblastic differentiation.

SiRNA mediated knockdown of DIO2 was found to accelerate mineralization in differentiating hMSC, in the presence or absence of BMP-7. DIO2 is a member of the iodothyronine deiodinase family, a group of enzymes whose members regulate thyroid hormone availability. DIO2 plays a critical role in the conversion of inactive pro-hormone T4 to its active form, T3 (Bianco et al., (2002), Endocr. Rev., 23:38-39). DIO2 is expressed by primary murine osteoblasts and MC3T3 cells (Williams et al., (2008), Bone, 43:126-34), and DIO2 activity has been identified in bone and bone marrow of adult mice (Guivea et al., (2005), Endocrinology, 146:195-200). The data described herein support Applicants' finding that DIO2 expression can serve to suppress MSC osteoblastic differentiation.

Applicants unexpectedly observed, an early, transient, attenuation of the cell cycle following BMP-7 treatment of hMSC. Osteoblastic differentiation is tightly linked to cell cycle regulation through Runx2/Cbfa-1 expression, which peaks during G₁ and exerts anti-proliferative effects (Galindo et al., (2005), J. Biol. Chem., 280:20274-85). In addition, Runx2/Cbfa-1 interacts with retinoblastoma protein to co-activate osteoblast-associated gene promoters (Thomas et al., (2001), Mol. Cell., 8:303-16. BMP-4 induces G₁ arrest during early osteoblastic differentiation of MC3T3-E1 cells (Mogi et al., (2003), Biol Chem., 278:47477-82). Collectively, Applicants' data indicate a heretofore unreported role for BMP-7 in the arrest of cell cycle progression that occurs concomitant to the initiation of cellular differentiation.

Applicants' data also show that BMP-7 significantly inhibits cytokine production and secretion in hMSC (see Example 6 below). This activity can lead to modulation of osteoclast activity by BMP-7 during bone formation, as several cytokines down-regulated in this study promote osteoclast precursor recruitment, osteoclastogenesis and osteoclastic bone resorption (Ishimi et al., (1990), J. Immunol., 145:3297-303; Sato et al., (1995), J Cell Physiol., 164:197-204; Grano et al., (1996), Proc. Natl. Acad. Sci. USA, 93:7644-8; Li et al., (2007), J. Biol. Chem., 282:33098-106; Grassi et al., (2004), J. Cell Physiol., 199:244-51; Nakagawa et al., (2000), FEBS Lett., 473:161-4; Horowitz et al., (2001), Cytokine Growth Factor Rev., 12:9-18). BMP-7 mediated cytokine down-regulation can also serve to control localized immune responses. Immunosuppressive properties of BMP-7 have been documented in a variety of disease conditions linked to aberrant immune responses (Chubinskaya et al., (2007), Int. Orthop., 31(6):773-81; Gould et al., (2002), Kidney Int., 61:51-60; Maric et al., (2003), J. Cell Physiol., 196:258-64; Hruska et al., (2000), Am. J. Physiol. Renal Physiol., 196:258-64). Interestingly, while proinflammatory cytokines including IL-6 initiate signaling cascades required for bone regeneration following injury, significant evidence supports the use of targeted immunomodulatory agents to control inflammation during fracture healing (Mountziaris et al., (2008), Tissue Neg. Part B. Rev., 14:179-86).

Applicants' data also demonstrate modulation of CHI3L1 expression by BMPs, which has not previously been reported (see Example 7 below). Osteoblastic expression of CHI3L1 has been described in osteophytic bone (Connor et al., (2000), Osteoarthritis Cartilage, 8:87-95), but the role of this protein in normal human osteoblastic function was heretofore undefined. CHI3L1 is elevated in many joint diseases Connor et al., (2000), Osteoarthritis Cartilage, 8:87-95; Johansen et al., (1999), Rheumatology, 38:618-26; Trudel et al., (2007), Clin. Orthop. Relat. Res., 456:92-7), and has been shown to stimulate production of ECM components in murine, rabbit and guinea pig chondrocytes (Jacques et al., (2007), Osteoarthris Cartilage, 15:138-46; De Ceuninck et al., (2001), Biochem. Biophys. Res. Commun., 285:926-31). BMP-7 mediated down-regulation of CHI3L1 under osteogenic conditions can indicate transition from cartilage to bone deposition during endochondral bone formation.

Applicants also investigated expression of other BMP and GDF genes, and BMP inhibitors by interrogation of the Affymetrix dataset (see Example 8 below). Confirmation studies demonstrated that several BMP inhibitors, including NOG, BAMBI, GREM1 and GREM2, were significantly up-regulated by BMP-7 (see Example 9 below). Induction of NOG in BMP-7 treated cells far exceeded that of all other inhibitors, suggesting that noggin may act as the principal negative regulator of BMP-7 function during human osteoblastic differentiation. Applicants' data is in agreement with that published by Gazzerro et al. where the authors describe a dose-dependent induction of noggin following BMP-2, BMP-4 or BMP-6 treatment of rat osteoblasts (Gazzerro et al., (1998), J. Clin. Invest., 102:2106-14).

BMP-7 treatment of hMSC resulted in a down-regulation of the chondrogenic growth factor GDF5 in differentiating hMSC (see Example 8 below). Inhibition of GDF5 by BMP-7 under osteogenic conditions can contribute to the shift from chondrogenesis to osteoblastogenesis that occurs during endochondral bone formation (Erlacher et al., (1998), J. Bone Miner Res., 13:383-92). BMP-7 up-regulated BMP-2 mRNA expression in primary hMSC, but did not induce expression of BMP-7 or that of other BMPs. BMP-2 is also induced in primary hMSC by other osteogenic BMPs, including BMP-2, BMP-4 and BMP-6, while none of these BMPs induce BMP-7. In mice, absence of BMP-2 expression results in a failure of fracture healing in limbs, despite some detectable expression of BMP-7 (Tsuji et al., (2006), Nature Genetics, 38:1424-9). Applicants have demonstrated inhibition of BMP-2 expression did not block the BMP-7 mediated development of an osteoblast phenotype. Significantly, these data indicate that endogenous BMP-2 is not required for in vitro osteoblastic differentiation of hMSC in the presence of exogenous BMP-7.

In short, the Examples set forth herein confirm a heretofore unreported effect of BMP-7 on the bioactivity of several specific genes and/or gene expression products during early osteoblastic differentiation of primary hMSC. Furthermore, these examples confirm a significant utility for an exemplary BMP, BMP-7, as a therapeutic agent and tissue engineering tool.

### Bone Morphogenetic Proteins

BMP-7 is a bone morphogenetic protein (“BMP”). Bone morphogenetic proteins belong to the TGF-β superfamily. The TGF-β superfamily proteins are cytokines characterized by six-conserved cysteine residues. The human genome contains about 42 open reading frames encoding TGF-β superfamily proteins. The TGF-β superfamily proteins can at least be divided into the BMP subfamily and the TGF-β subfamily based on sequence similarity and the specific signaling pathways that they activate.

The BMP subfamily includes, but is not limited to, BMP-2, BMP-3 (osteogenin), BMP-3b (GDF-10), BMP-4 (BMP-2b), BMP-5, BMP-6, BMP-7 (osteogenic protein-1 or OP-1), BMP-8 (OP-2), BMP-8B (OP-3), BMP-9 (GDF-2), BMP-10, BMP-11 (GDF-11), BMP-12 (GDF-7), BMP-13 (GDF-6, CDMP-2), BMP-15 (GDF-9), BMP-16, GDF-1, GDF-3, GDF-5 (CDMP-1, MP-52), and GDF-8 (myostatin). BMPs are also present in other animal species. Furthermore, there is allelic variation in BMP sequences among different members of the human population, and there is species variation among BMPs discovered and characterized to date.

The TGF-β subfamily includes, but is not limited to, TGFs (e.g., TGF-β31, TGF-β2, and TGF-β3), activins (e.g., activin A) and inhibins, macrophage inhibitory cytokine-1 (MIC-1), Mullerian inhibiting substance, anti-Mullerian hormone, and glial cell line derived neurotrophic factor (GDNF). As used herein, “TGF-β subfamily,” “TGF-βs,” “TGF-β ligands” and grammatical equivalents thereof refer to the TGF-β subfamily members, unless specifically indicated otherwise.

The TGF-β superfamily is in turn a subset of the cysteine knot cytokine superfamily. Additional members of the cysteine knot cytokine superfamily include, but are not limited to, platelet derived growth factor (PDGF), vascular endothelial growth factor (VEGF), placenta growth factor (PIGF), noggin, neurotrophins (BDNF, NT3, NT4, and βNGF), gonadotropin, follitropin, lutropin, interleukin-17, and coagulogen.

Publications disclosing these sequences, as well as their chemical and physical properties, include: BMP-7 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-4, (WO88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)) The above publications are incorporated herein by reference.

As used herein, “TGF-β superfamily member” or “TGF-β superfamily protein,” means a protein known to those of ordinary skill in the art as a member of the Transforming Growth Factor-β (TGF-β superfamily. Structurally, such proteins are homo or heterodimers expressed as large precursor polypeptide chains containing a hydrophobic signal sequence, an N-terminal pro region of several hundred amino acids, and a mature domain comprising a variable N-terminal region and a highly conserved C-terminal region containing approximately 100 amino acids with a characteristic cysteine motif having a conserved six or seven cysteine skeleton. These structurally-related proteins have been identified as being involved in a variety of developmental events.

The term “morphogenic protein” refers to a protein belonging to the TGF-β superfamily of proteins which has true morphogenic activity. For instance, such a protein is capable of inducing progenitor cells to proliferate and/or to initiate a cascade of events in a differentiation pathway that leads to the formation of cartilage, bone, tendon, ligament, neural or other types of differentiated tissue, depending on local environmental cues. Accordingly, morphogenic proteins can behave differently in different surroundings. Morphogenic proteins can also be homodimeric or heterodimeric.

“Osteogenic protein (OP)” refers to a morphogenic protein that is also capable of inducing a progenitor cell to form cartilage and/or bone. The bone can be intramembranous bone or endochondral bone. Most osteogenic proteins are members of the BMP subfamily and are thus also BMPs. However, the converse can not be true. For example, a BMP identified by DNA sequence homology or amino acid sequence identity must also have demonstrable osteogenic or chondrogenic activity in a functional bioassay in order to be an osteogenic protein. Appropriate bioassays are well known in the art; a particularly useful bioassay is the heterotopic bone formation assay (see, U.S. Pat. No. 5,011,691; U.S. Pat. No. 5,266,683, for example).

Structurally, BMPs are dimeric cysteine knot proteins. Each BMP monomer comprises multiple intramolecular disulfide bonds. An additional intermolecular disulfide bond mediates dimerization in most BMPs. BMPs may form homodimers. Some BMPs may form heterodimers. BMPs are expressed as pro-proteins comprising a long pro-domain, one or more cleavage sites, and a mature domain. The pro-domain is believed to aid in the correct folding and processing of BMPs. Furthermore, in some but not all BMPs, the pro-domain may noncovalently bind the mature domain and may act as an inhibitor (e.g., Thies et al. (2001) Growth Factors 18:251-259).

BMPs are naturally expressed as pro-proteins comprising a long pro-domain, one or more cleavage sites, and a mature domain. This pro-protein is then processed by the cellular machinery to yield a dimeric mature BMP molecule. The pro-domain is believed to aid in the correct folding and processing of BMPs. Furthermore, in some but not all BMPs, the pro-domain may noncovalently bind the mature domain and may act as a chaperone, as well as an inhibitor (e.g., Thies et. al. (2001) Growth Factors, 18:251-259).

BMPs belong to the BMP subfamily of the TGF-β superfamily of proteins defined on the basis of DNA homology and amino acid sequence identity. As described herein, a protein belongs to the BMP subfamily when it has at least 50% amino acid sequence identity with a known BMP subfamily member within the conserved C-terminal cysteine-rich domain that characterizes the BMP subfamily. Members of the BMP subfamily can have less than 50% DNA or amino acid sequence identity overall. As used herein, the term “BMP” further refers to proteins which are amino acid sequence variants, domain-swapped variants, and truncations and active fragments of naturally occurring bone morphogenetic proteins, as well as heterodimeric proteins formed from two different monomeric BMP peptides, such as BMP-2/7; BMP-4/7: BMP-2/6; BMP-2/5; BMP-4/7; BMP-4/5; and BMP-4/6 heterodimers. Suitable BMP variants and heterodimers include those set forth in US 2006/0235204; WO 07/087,053; WO 05/097825; WO 00/020607; WO 00/020591; WO 00/020449; WO 05/113585; WO 95/016034 and WO93/009229.

BMP-7 utilized in methods and compositions of the invention, in some embodiments, includes variants of BMP-7. For example, in some embodiments, BMP-7 which retain at least 50% of wild type BMP-7 activity in one or more cell types, as determined using an appropriate assay described below is contemplated by the invention. For example, in some embodiments, BMP-7 includes variants maintaining 75%, 80%, 85%, 90% or 95% of wild type activity. In some embodiments, BMP-7 includes variants containing insertions, deletions, and/or substitutions at the N-terminus, C-terminus, or internally and/or may have at least 1, 2, 3, 4, 5, 6, 7, 8, 9, 10 or more different residues.

Variant BMP-7 utilized in methods and compositions of the invention, in some embodiments, maintain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the corresponding wild-type BMP-7 protein sequence.

Variant BMP-7 utilized in methods and compositions of the invention, in some embodiments, maintain at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with the conserved cysteine domain of the C-terminal region of the corresponding wild-type BMP-7 protein sequence.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions×100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al. (1990) J. Mol. Biol. 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used.

### DIO2

Based on Applicant's observation that DIO2 expression suppresses osteoblastic differentiation, one aspect of the invention involves manipulating the expression of DIO2 or the activity of its expressed protein to affect osteoblastic differentiation.

Accordingly, in one embodiment, the invention includes methods for promoting endochondral bone formation that involve down-regulating or inhibiting the expression of the DIO2 gene. Such methods promote mineralization of differentiating osteoblasts. Down-regulating or inhibiting the expression of the DIO2 gene can be achieved through standard methods known in the art, such as, for example, through an siRNA or other interfering RNA targeting the transcription product of the DIO2 gene, or through a structural or functional analog of an interfering RNA or through other methods that would silence gene transcription.

In another embodiment, the invention includes methods for promoting endochondral bone formation that involve controlling, reducing, or suppressing the activity of the expression product of the DIO2 gene, i.e., the expressed protein (which is known as Type II iodothyronine deiodinase (DIO2 protein)). This promotes mineralization of the osteoblasts. Controlling, reducing or suppressing the activity of the DIO2 protein can be achieved through many of the standard methods known in the art, such as, for example, through an agent that reduces the activity of DIO2 protein, for example a DIO2 antagonist or inhibitor, whether natural or synthetic. For example, the inhibitor is iopanoic acid, or an analog of iopanoic acid.

In another embodiment, an effective amount of an agent which down-regulates or inhibits the expression of the DIO2 gene is administered in conjunction with BMP-7, a BMP-7 agonist, or BMP-7 and a BMP-7 antagonist to induce osteoblastogenesis, but not mineralization of the resulting osteoblasts. The non-mineralized osteoblasts are permitted to accumulate. The agent's effect is then neutralized or reversed to promote mineralization of accumulated osteoblasts and endochondral bone formation. According to one embodiment of the invention, an effective amount of the agent is added to cells, for example, MSCs, ex vivo, for example, in vitro, and the cells accumulate in vitro. In one embodiment, the neutralizing step is performed in vitro. For example, the neutralizing step is performed prior to cells being implanted in a patient. In another embodiment, the neutralizing step is performed once the cells are implanted in the patient. As a result of the neutralizing or reversing step, osteoblasts are mineralized and endochondral bone formation occurs. In another embodiment, example, the cells are implanted in the patient at a site in need of bone repair. The site can be, for example, the location of a bone fracture, chip, or other bone injury.

In another embodiment, an effective amount of an agent which reduces or blocks the activity of the DIO2 protein is administered in conjunction with BMP-7, a BMP-7 agonist, or BMP-7 and a BMP-7 antagonist to induce osteoblastogenesis, but not mineralization of the resulting osteoblasts. The non-mineralized osteoblasts are permitted to accumulate. The agent's effect is then neutralized or reversed to promote mineralization of accumulated osteoblasts and endochondral bone formation. According to one embodiment of the invention, an effective amount of the agent is added to cells, for example, MSCs, ex vivo, for example, in vitro, and the cells accumulate in vitro. In one embodiment, the neutralizing step is performed in vitro. For example, the neutralizing step is performed prior to cells being implanted in a patient. In another embodiment, the neutralizing step is performed once the cells are implanted in a patient. As a result of the neutralizing step, osteoblasts are mineralized and endochondral bone formation occurs. In another embodiment, the cells are implanted in the patient at a site in need of bone repair. The site can be, for example, the location of a bone fracture, chip, or other bone injury.

The neutralizing step of the above methods can be performed by metabolic depletion or exhaustion of the agent over time. In another embodiment, the neutralizing step is performed by administering a neutralizing agent in an effective amount to block or deactivate the agent which reduces or blocks the activity of the DIO2 protein or the expression of the DIO2 gene. For example, a neutralizing agent includes T3, an agonist of the DIO2 protein, and/or an agonist of the DIO2 gene expression.

According to another embodiment, a further step in the above-identified methods includes administering an effective amount of an agent which reduces or blocks the activity of Noggin and/or the Noggin gene. The administration an agent which reduces or blocks the activity of Noggin and/or the Noggin gene can occur, for example, in one embodiment, prior to the reversing/neutralizing step above. In other embodiment, the administration an agent which reduces or blocks the activity of Noggin and/or the Noggin gene occurs at the same time as the administration of the agent that reduces or blocks the activity of the DIO2 protein or the DIO2 gene expression. In another embodiment, the administration of the agent which reduces or blocks the activity of Noggin and/or the Noggin gene occurs while allowing accumulation of non-mineralized osteoblasts.

According to these methods, the effective amount of BMP-7 may be exogenous BMP-7 or it may be endogenous BMP-7 in the patient.

According to another embodiment, MSC used according to the invention can be allogeneic or autogenic MSC. For example, in one embodiment, allogeneic MSC are implanted in patient and acted on according to methods of the invention, while in a further embodiment, the methods of the present invention are performed on the patient's own MSC endogenously. In an other embodiment, allogeneic or autogenic MSC are treated according to methods of the invention prior to implantation in the patient.

In another embodiment according to the invention, an effective amount of an agent which reduces or blocks the activity of the DIO2 protein and an effective amount of an agent which down-regulates or inhibits the expression of the DIO2 gene is administered in conjunction with BMP-7, a BMP-7 agonist, or BMP-7 and a BMP-7 antagonist to induce osteoblastogenesis, but not mineralization of the resulting osteoblasts.

The invention also includes a method for preparing non-mineralized, differentiated osteoblasts. The method requires contacting mesenchymal stem cells with an effective amount of BMP-7 and an effective amount of an agent which reduces or blocks the activity of the DIO2 gene or the DIO2 protein. The MSCs undergo osteoblastogenesis to form differentiated osteoblasts which are non-mineralized. The MSCs are human in one embodiment. In one embodiment, the BMP-7 is endogenous to a patient. In another embodiment, the BMP-7 is from a source exogenous to a patient. In another embodiment, MSCs are implanted into a patient prior to differentiation. In another embodiment, the differentiated osteoblasts are implanted into a patient. In another embodiment, the contacting step occurs ex vivo to the patient, for example, in vitro. In yet another embodiment, the contacting step occurs in the patient. For example, the patient is a human. In yet another embodiment, the MSCs are endogenous to the patient and the contacting step occurs in the patient. For example, the patient is administered between 100 μg to 20 mg when the MSCs are contact with BMP-7 in the patient. In another embodiment, when the MSCs are contacted with BMP-7 in vitro, the amount of BMP-7 is between 10 ng/mL to 10 μg/ml.

The invention further includes another method for preparing non-mineralized differentiated osteoblasts. The method includes contacting MSCs with an effective amount of BMP-7 and an effective amount of an agent which reduces or blocks the activity of a T3 molecule. The cells undergo osteoblastogenesis to form differentiated osteoblasts which are non-mineralized. The MSCs, in one embodiment, are human. In another embodiment, MSCs are implanted into a patient prior to differentiation. In another embodiment, the differentiated osteoblasts are implanted into a patient. The patient can be human. In a further embodiment, the effective amount of BMP-7 is endogenous to the patient, while in another embodiment, the BMP-7 is from a source exogenous to the patient. In a further embodiment, the contacting step occurs ex vivo to the patient, for example, in vitro. In yet another embodiment, the contacting step occurs in the patient. In yet another embodiment, the MSCs are endogenous to the patient and the contacting step occurs in the patient. For example, the patient is administered between 100 μg to 20 mg when the MSCs are contact with BMP-7 in the patient. In another embodiment, when the MSCs are contacted with BMP-7 in vitro, the amount of BMP-7 is between 10 ng/mL to 10 μg/ml.

The invention includes a further method for preparing non-mineralized, differentiated osteoblasts which includes the step of contacting MSC with an effective amount of BMP-7 and an effective amount of a T4 antagonist or analog which prevents the formation of T3. The cells undergo osteoblastogenesis to form differentiated osteoblasts which are non-mineralized. The MSCs, in one embodiment, are human. In another embodiment, MSCs are implanted into a patient prior to differentiation. In another embodiment, the differentiated osteoblasts are implanted into a patient. The patient can be human. In a further embodiment, the effective amount of BMP-7 is endogenous to the patient, while in another embodiment, the BMP-7 is from a source exogenous to the patient. In a further embodiment, the contacting step occurs ex vivo to the patient, for example, in vitro. In yet another embodiment, the contacting step occurs in the patient.

The invention includes a method for promoting osteoblastogenesis of non-mineralized osteoblasts, differentiated osteoblasts which includes the step of contacting MSC with an effective amount of BMP-7 and an effective amount of an agent which reduces or blocks the activity of DIO2 protein and/or the expression of the DIO2 gene. The cells undergo osteoblastogenesis to form differentiated osteoblasts which are non-mineralized. The MSCs, in one embodiment, are human. In another embodiment, MSCs are implanted into a patient prior to differentiation. In another embodiment, the differentiated osteoblasts are implanted into a patient. The patient can be human. In a further embodiment, the effective amount of BMP-7 is endogenous to the patient, while in another embodiment, the BMP-7 is from a source exogenous to the patient. In a further embodiment, the contacting step occurs ex vivo to the patient, for example, in vitro. In yet another embodiment, the contacting step occurs in the patient.

By “non-mineralized” is meant that substantially all of a population of differentiated osteoblasts is not mineralized. According to the invention, methods of that promote osteoblastogenesis but not mineralization may result in some mineralized cells, but the substantially all of the cells in a given population will be non-mineralized.

The invention also includes compositions that reduce or block the activity of the DIO2 protein and/or the expression of the DIO2 gene. Such compositions have the effect of promoting endochondral bone formation. For example, in one embodiment, the invention provides a composition including BMP-7 and an agent which blocks the activity of the DIO2 protein and/or the DIO2 gene expression. In one embodiment, the BMP-7 is human BMP-7. In another embodiment, the agent is an inhibitor which blocks the activity of the DIO2 protein such as iopanoic acid or a structural or functional analog of iopanoic acid, or other non-natural or natural chemical inhibitor capable of reducing or blocking the activity of the DIO2 protein. In another embodiment, the agent is an inhibitor of the DIO2 gene such as siRNA or other interfering RNA, structural or functional analogs of interfering RNA, or a non-natural or naturally occurring non-chemical or chemical inhibitor.

In yet another embodiment, the invention includes a pharmaceutical composition including BMP-7 and/or a functional analog or mimetic or agonist thereof and an agent which reduces or blocks the activity of a T3 molecule. Such a composition has the effect of promoting endochondral bone formation. In a further embodiment, the composition includes an antagonist of noggin or an agent that effectively reduces or eliminates expression of the noggin gene.

In yet another embodiment, the invention includes a pharmaceutical composition including BMP-7 and a T4 analog or antagonist which prevents the formation of T3. In one embodiment, the BMP-7 is human BMP-7. Such a composition has the effect of promoting endochondral bone formation. In a further embodiment, the composition includes an antagonist of noggin or an agent that effectively reduces or eliminates expression of the noggin gene.

In yet another embodiment, the invention includes a composition comprising stem cells, BMP-7 or an agonist or analog thereof, and an antagonist of the DIO2 protein and/or DIO2 gene expression and/or T3. Such a composition has the effect of promoting endochondral bone formation. Such a composition has the effect of promoting endochondral bone formation. In a further embodiment, the composition includes an antagonist of noggin or an agent that effectively reduces or eliminates expression of the noggin gene.

### FGFR3, ADAMTS9, HEY1, HAS3, and MFI2

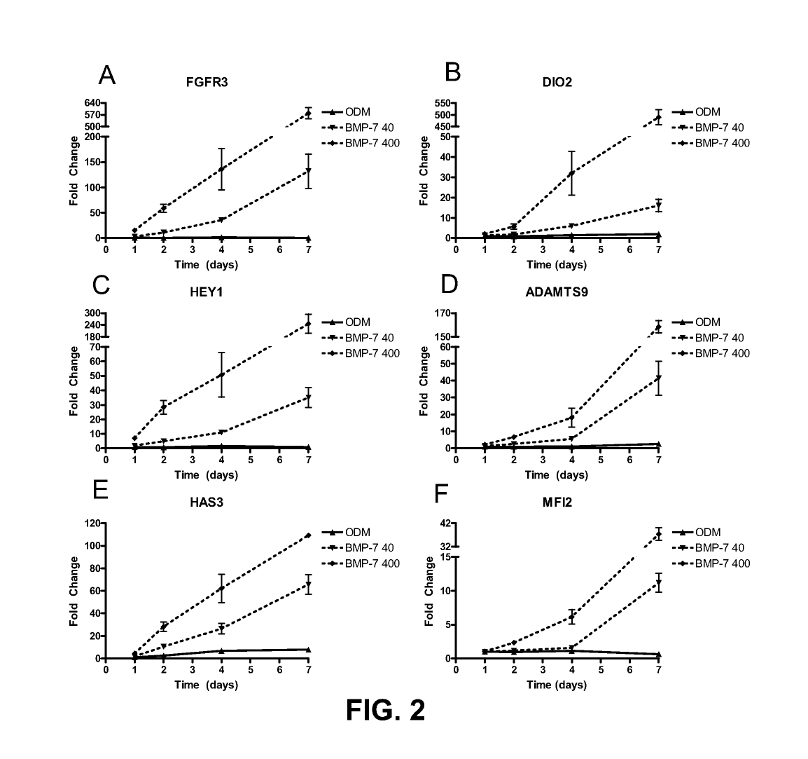

As evidenced by Applicants' data, expression of FGFR3, ADAMTS9, HEY1, HAS3, and MFI2 is mediated by BMP-7 during early osteoblastic differentiation of primary human MSCs. These genes are subject to up-regulation in the presence of BMP-7 (see FIGS. 2A-F).

In one aspect, the invention includes methods for promoting endochondral bone formation. According to one embodiment, endochondral bone formation is promoted or enhanced by inducing or up-regulating the expression of one or more of the FGFR3, ADAMTS9, HEY1, HAS3, and MFI2 genes. In one embodiment, inducing or up-regulating the activity of these genes involves administering BMP-7 to a cell in an amount effective to up-regulate the expression of the gene. As used herein, in one embodiment, “up-regulate” can mean to increase the expression level of a gene beyond the normal or base level of gene expression in a similarly situated control cell absent the presence of the up-regulating agent. In another embodiment, “p-regulate” can also mean to increase the expression level of a gene beyond the level of gene expression prior to administering the up-regulating agent. In another embodiment, “up-regulate” can also mean to increase the stability of mRNA encoding a protein or the protein itself, thereby increasing the expression of the gene or activity of the protein.

In another embodiment of the invention, a method for promoting endochondral bone formation includes enhancing the activity of a protein encoded by one of the FGFR3, ADAMTS9, HEY1, HAS3, or MFI2 genes. For example, in one embodiment, the activity of the protein is enhanced by administering an agonist of the protein. For example, in the case of the FGFR3 protein, an agonist, such as FGF that is specific for binding to FGFR3, is administered to increase the activity of FGFR3, although any known FGFR3 agonist would be suitable for administration to increase the activity of the FGFR3 receptor protein.

In the case of ADAMTS9 protein, an agonist of this protein's activity can be administered to enhance ADAMTS9 activity and promote endochondral bone formation. Further, in another embodiment, molecules that promote the activation of ADAMTS9 can also be administered to enhance ADAMTS9 activity

In the case of HEY1 protein, an agonist of this protein's activity can be administered to enhance HEY1 activity and promote endochondral bone formation.

In the case of HAS3 protein, an agonist of this protein's activity can be administered to enhance HAS3 activity and promote endochondral bone formation.

In the case of MFI2 protein, an agonist of this protein's activity can be administered to enhance MFI2 activity and promote endochondral bone formation.

In a further embodiment, a method for enhancing or promoting the formation of endochondral bone includes up-regulating or enhancing the expression of one or more of the FGFR3, ADAMTS9, HEY1, HAS3, and MFI2 genes and the gene's expressed protein activity by administering BMP-7 in combination with an agonist of one or more of FGFR3, ADAMTS9, HEY1, HAS3, and MFI2.

According to one embodiment, BMP-7 in combination with an agonist of one or more of the FGFR3, ADAMTS9, HEY1, HAS3, and MFI2 proteins is administered to a patient in need of endochondral bone formation, for example, to heal or repair diseased, damaged or missing endochondral bone in the patient. In another embodiment, BMP-7 in combination with an agonist of one or more of the FGFR3, ADAMTS9, HEY1, HAS3, and MFI2 proteins is contacted with a population of mesenchymal stem cells to induce differentiation of those cells. The MSCs are subsequently implanted in a patient in need of endochondral bone formation. In one embodiment, the MSCs are implanted in or administered to the patient prior to completion of osteoblastogenesis. In another embodiment, non-mineralized osteoblasts resulting from the differentiating MSC population are implanted in or administered to the patient. In one embodiment, the patient is a mammal, while in a preferred embodiment, the patient is a human.

According to another aspect, the invention includes compositions for enhancing or promoting the growth of endochondral bone. For example, in one embodiment, a composition for enhancing or promoting the growth of endochondral bone includes BMP-7 and an agonist of FGFR3. For example, in one embodiment, a composition for promoting the growth of endochondral bone includes BMP-7 and an FGF that is specific for binding to FGFR3. In another embodiment, a composition for enhancing or promoting the growth of endochondral bone includes BMP-7 and an agonist of ADAMTS9. In yet another embodiment, a composition for enhancing or promoting the growth of endochondral bone includes BMP-7 and an agonist of HEY1. In a further embodiment, a composition for enhancing or promoting the growth of endochondral bone includes BMP-7 and an agonist of HAS3. In an even further embodiment, a composition for enhancing or promoting the growth of endochondral bone includes BMP-7 and an agonist of MFI2.

Compositions of the invention for enhancing or promoting the growth of endochondral bone may also include combinations of agonists of the proteins encoded by the FGFR3, ADAMTS9, HEY1, HAS3, and MFI2 genes. For example, in one embodiment, a composition for enhancing or promoting the growth of endochondral bone includes BMP-7 and agonists of at least two of the FGFR3, ADAMTS9, HEY1, HAS3, or MFI2 proteins. In a further embodiment, a composition for enhancing or promoting the growth of endochondral bone includes BMP-7 and agonists of at least three of the FGFR3, ADAMTS9, HEY1, HAS3, and MFI2 proteins. In a further embodiment, a composition for enhancing or promoting the growth of endochondral bone includes BMP-7 and agonists of at least four of FGFR3, ADAMTS9, HEY1, HAS3, and MFI2 proteins. In yet a further embodiment, a composition for enhancing or promoting the growth of endochondral bone includes BMP-7 and agonists of FGFR3, ADAMTS9, HEY1, HAS3, and MFI2.

Compositions of the invention for enhancing or promoting the growth of endochondral bone may also include cells. For example, in one embodiment, a composition for enhancing or promoting the growth of endochondral bone includes a population of mesenchymal stem cells, BMP-7, and an agonist of one or more of the FGFR3, ADAMTS9, HEY1, HAS3, or MFI2 proteins. In another embodiment, a composition for enhancing or promoting the growth of endochondral bone includes a population of non-mineralized osteoblasts, BMP-7, and an agonist of one or more of the FGFR3, ADAMTS9, HEY1, HAS3, or MFI2 proteins.

### CHI3L1

In yet another embodiment, the invention includes a method for modulating endochondral bone formation which includes the step of down-regulating the expression of the CHI3L1 gene and/or reducing or inhibiting the activity of CHI3L1 protein (chitinase 3-like protein 1) to promote bone deposition. Down-regulating the expression of the gene can be achieved by administering an effective amount of BMP-7, while reducing or inhibiting the activity of CHI3L1 protein can be achieved by administering a CHI3L1 antagonist. According to the method, bone deposition follows CHI3L1 down-regulation. According to one embodiment of the invention, BMP-7 and an antagonist of CHI3L1 are administered to a population of MSC in vitro and then the cell population is implanted in or administered to a patient to permit endochondral bone formation in the patient. The MSC can be allogeneic or autogenic to the patient to whom the cells are administered. In another embodiment, BMP-7 and an antagonist of CHI3L1 are administered to a patient to promote endochondral bone formation by differentiation of MSCs endogenous to the patient.

Accordingly, in a further embodiment, the invention includes a composition effective in modulating the formation of endochondral bone comprising BMP-7 and an antagonist of the CHI3L1 protein. In a further embodiment, the invention includes a composition effective in modulating the formation of endochondral bone comprising BMP-7, an antagonist of the CHI3L1 protein, and mesenchymal stem cells. The MSC can be allogeneic or autogenic to a patient to whom the composition is administered.

### Cytokine Down-Regulation

As described in Example 6 below, BMP-7 administration has the effect of down-regulated cytokine expression in MSC. Several of the cytokines shown by the data to be down-regulated by BMP-7 administration are known to promote osteoclast precursor recruitment, osteoclastogenesis, and osteoclastic bone resorption. Accordingly, the invention includes a method of tissue engineering including the step of providing BMP-7 or a BMP-7 analog or mimetic or agonist thereof in an amount effective to down-regulate osteoclastic events including chemokine or cytokine-induced osteoclastogenesis.

### Cartilage Formation

Mesenchymal stem cells can differentiate into osteocytes or chondrocytes. Accordingly, arresting the differentiation of MSC in a state prior to mineralization may permit methods whereby MSCs can be manipulated to form cartilage rather than bone. Accordingly, in one embodiment, the invention includes methods for promoting cartilage formation that include down-regulating genes that promote mineralization of osteoblasts thereby preventing mineralization and providing the opportunity to arrest cell differentiation prior to mineralization.

Accordingly, in one embodiment, the invention includes methods for promoting cartilage formation that include administering one or more agents that enhances expression of the DIO2 gene and/or activity of the DIO2 protein. For example, in one embodiment, one or more agents that enhances expression of the DIO2 gene and/or activity of the DIO2 protein is contacted with a population of MSC to block mineralization. The method can further include the step of administering a protein that promotes chondrogenesis such as GDF-5. The cells can be implanted in a patient after being contacted with GDF-5 or the cells can be implanted in a patient and contacted with GDF-5 at the time of or after implantation.

### Administration

Administration of agents and compositions described herein according to the various methods of the invention may be achieved according to a variety of methods. For example, the agents and compositions of the invention can be administered by any suitable means, e.g., parenteral, intravenous, subcutaneous, intramuscular, intraorbital, ophthalmic, intraventricular, intracranial, intracapsular, intraspinal, intracistemal, intraperitoneal, buccal, rectal, vaginal, intranasal or aerosol administration. Administration may be local, i.e., directed to a specific site, or systemic. Administration may also be effected by, but not limited to, direct surgical implantation, endoscopy, catheterization, or lavage. If applied during surgery, the compositions of the invention may be flowed onto the tissue, sprayed onto the tissue, painted onto the tissue, or any other means within the skill in the art. Alternatively, compositions of the invention applied during surgery may be provided in a putty, paste, or gel form, or incorporated into a suitable matrix for implantation. Further, compositions of the invention applied during surgery may be implanted in a patient at the site of a bone fracture or bone injury, into a spinal disc, into a joint, or any other location where endochondral bone formation is desired.

The compositions of the invention may be administered in or with an appropriate carrier or bulking agent including, but not limited to, a biocompatible oil such as sesame oil, hyaluronic acid, cyclodextrins, lactose, raffinose, mannitol, carboxy methyl cellulose, thermo or chemo-responsive gels, sucrose acetate isobutyrate.

As will be appreciated by those skilled in the art, the concentration of the compounds described in the compositions of the invention will vary depending upon a number of factors, including without limitation the dosage of the drug to be administered, the chemical characteristics (e.g., hydrophobicity) of the compounds employed, and the route of administration. The preferred dosage of drug to be administered also is likely to depend on variables including, but not limited to, the type and extent of a disease, tissue loss or defect, the overall health status of the particular patient, the relative biological efficacy of the compound selected, the formulation of the compound, the presence and types of excipients in the formulation, and the route of administration. The therapeutic molecules of the present invention may be provided to an individual where typical doses range from about 10 ng/kg to about 1 g/kg of body weight per day; with a preferred dose range being from about 0.1 mg/kg to 100 mg/kg of body weight, and with a more particularly preferred dosage range of 10-1000 μg/dose. In a particularly preferred embodiment, 10-1000 μg is a preferred dose of a BMP-7. The skilled clinician would appreciate that the effective doses of the present invention can be modified in light of numerous factors including, but not limited to, the indication, the pathology of the disease, and the physical characteristics of the individual. It is also clearly within the skill in the art to vary, modify, or optimize doses in view of any or all of the aforementioned factors.

### Example 1

### Materials and Methods

(a) Cell Culture

Primary human bone-marrow derived mesenchymal stem cells (hMSC) and hMSC culture media, including Mesenchymal Stem Cell Growth Medium (MSCGM) and Osteogenic Differentiation Medium (ODM), were purchased from Lonza (Walkersville, Md.). Cells were obtained from healthy donors between the ages of 18 and 45, and were tested by the manufacturer for multipotency down the osteogenic, chondrogenic and adipogenic lineages. In addition, cells were found to be positive by flow cytometry for expression of CD105, CD166, CD29, and CD44, and negative for CD14, CD34 and CD45. Cells were expanded in vitro and used for experimentation within four passages of the initial thaw.

(b) BMP Treatment

BMP-7 was prepared by Stryker Biotech as previously described (Sampath et al., (1992), J. Biol. Chem, 267:20352-62). ODM was prepared according to the manufacturer's instructions using the provided supplements of ascorbic acid and beta glycerophosphate but excluding the dexamethasone. BMP-7 was diluted in ODM to the indicated concentrations.

(c) Alkaline Phosphatase Activity Assays

Primary hMSC were seeded in MSCGM in 96-well dishes at 5×10³ cells per well. The following day, cells were treated with ODM alone or ODM supplemented with serial dilutions of BMP-7 from 4 μg/ml to 16 ng/ml. After 6 days of treatment, cells were lysed with 1% Triton-X (Sigma-Aldrich, St. Louise, Mo.). AP activity of each cell lysate was determined using pNPP Reagent (Moss Inc., Pasadena, Md.), incubated until significant color developed and read at A490 nm. 4-Nitrophenol (4-NP) produced per minute was determined relative to A490 readings of a standard curve generated using serial dilutions of 4-NP (Sigma-Aldrich, St. Louis, Mo.). AP Activity from each well was normalized to total protein, which was quantified using a BCA Protein Assay Kit (Pierce Biotechnology, Rockford, Ill.).

(d) Alizarin Red Mineralization Staining and Quantification of Calcium Content

Primary hMSC were seeded in MSCGM in 48-well dishes at 1.5×10⁴ cells per well. The following day, cells were treated with ODM or ODM containing the indicated dose of BMP-7. Media changes were performed every 3-4 days. Alizarin Red staining was performed at the indicated time points using an Osteogenesis Quantitation Kit (Chemicon International, Temecula, Calif.). Quantification of calcium content was performed using a Calcium (CPC) Liquicolor Kit (StanBio Laboratory, Boerne, Tex.). Calcium content was normalized to total protein using a BCA Protein Assay Kit (Pierce Biotechnology, Rockford, Ill.).

(e) Gene Expression Analysis by High Density Microarrays

Primary hMSC were seeded in MSCGM at 1.5×10⁴ cells per cm² in T-75 tissue culture flasks. Twenty four hours later, at approximately 70% confluence, cells were treated with ODM alone or ODM containing 500 ng/ml BMP-7. Five replicates of the BMP-7 treated cells and four replicates of the controls were harvested after 24 and 120 hours, and processed on Affymetrix® HG-U133 Plus 2.0 Arrays to evaluate gene expression across the entire human genome. Sample processing was performed by Asuragen, Inc. (Austin, Tex.) according to the company's standard operating procedures. Briefly, total RNA was isolated using ToTALLY RNA™ and used for preparation of biotin-labeled targets (cRNA) by standard RT-IVT methods using the MessageAmp™ II kit (Ambion Inc., Austin, Tex.). Labeled cRNA was fragmented and used for array hybridization. Arrays were washed and stained with Streptavidin-Phycoerythrein conjugate (SAPE) on an Affymetrix FS450 Fluidics station and scanned on an Affymetrix GCS 3000.

Data analysis was performed by Genome Explorations, Inc. (Memphis, Tenn.). Data were normalized using two different methods, the Affymetrix Statistical Algorithm MAS 5.0 (GCOS v1.4) and RMA (Lockhart et al., (1996), Nat Biotechnol., 14:1675-80; Liu et al., (2002), Bioinformatics, 18:1593-9; Hubbell et al., (2002), Bioinformatics, 18:1585-92; Irizarry et al., (2006), Bioinformatics, 22:789-94). Each normalized data set was subjected to ANOVA and independent t-tests at each time point (treated versus control) using the Benjamini-Hochberg FDR correction method. Differentially expressed genes (DEGs) were identified as having a corrected ANOVA p-value ≦0.01, an absolute fold change ≧2, and a t-test p-value of ≦0.01 in at least one pair-wise comparison. Probe sets common to both DEG lists were identified by Boolean intersection and used as the final data set for further analysis.

Unsupervised hierarchical clustering was performed by UPGMA (Unweighted Pair-Group method using Arithmetic Averages) on row mean centered log₂ transformed RMA normalized signal values using Pearson correlation distance as the similarity metric. Specific interrogations of the data set, including generation of BMP inhibitor, BMP and GDF lists, were performed with the RMA normalized data without filtering for expression level or fold change. Gene annotation, gene ontology information and biochemical pathway information were obtained from the National Center for Biotechnology Information (www.ncbi.nlm.nih.gov), NetAffx (ww.affymetrix.com), the Gene Ontology Consortium (http://amigo.geneontology.org), the Kyoto Encyclopedia of Genes and Genomes (www.genome.jp/kegg), and WebGestalt (http://bioinfo.vanderbilt.edu/webgestalt) (Zhang et al., (2005), Nucleic Acids Res, 33:W741-8). Significant enrichment of specific Gene Ontology (GO) categories or KEGG pathways was estimated by hypergeometric tests (p-values ≦0.05) using the U133 Plus 2.0 array content as the reference set.

(f) Gene Expression Analysis by Quantitative RT-PCR

RNA was isolated using the TurboCapture 96 mRNA Kit (Qiagen, Valencia, Calif.) according to the manufacturer's instructions. Reverse transcription was performed using 40 units of M-MLV Reverse Transcriptase (Promega, Madison, Wis.) in a buffer containing 20 mM Tris-HCl, 50 mM KCl, 5 mM MgCl₂, 500 μM each dNTP (Invitrogen, Carlsbad, Calif.) and 5 ng/μl Random Primers (Promega, Madison, Wis.). Reverse transcription was carried out at 23° C. for 10 minutes, 42° C. for 50 minutes followed by a 5 minute inactivation step at 85° C. All reagents and instrumentation for gene expression analysis were obtained from Applied Biosystems (ABI, Foster City, Calif.). Quantitative PCR was carried out using a 7900HT Fast Real-Time PCR System and pre-designed TaqMan Gene Expression Assays according to the manufacturer's specifications. Target gene expression was measured using the standard curve method of relative quantification, according to Applied Biosystems' recommended procedure.

(g) Transient Gene Knockdown

Stealth RNAi (Invitrogen, Carlsbad Calif.) was used to target DIO2, HEY1, HAS3, MFI2, BMPR1A and ACVR1 to name but a few genes. A BMP-2 siRNA On-Targetplus SMART pool was purchased from Dharmacon (Lafayette, Colo.). Chemistry matched negative controls (non-targeted sequences) were utilized as controls to confirm the specificity of each targeted knockdown. Pools of BMPR1A and ACVR1, two receptors critical to signaling by BMP-7 (Layery et al., (2008), J. Chem. Biol., 283:20948-58) were used as positive controls to inhibit osteoblastic differentiation. Primary hMSC were transfected with siRNA using a Nucleofector II (Amaxa Biosystems, Gaithersburg, Md.) and employing the manufacturer's hMSC Kit. A total of 4 μg of siRNA was delivered to 5×10⁵ hMSC. Cells were seeded at approximately 1.5×10⁴ cells per well in 48-well dishes and cultured in MSCGM for 48 hours to allow down-regulation of gene targets. Cells were then treated with BMP-7 at the indicated doses and for the indicated time periods.

(h) Cell Cycle Analysis by Flow Cytometry

Flow cytometry studies were performed at Southern Research Institute (Birmingham, Ala.). Primary hMSC were plated in MSCGM in 35-mm dishes at 1.5×10⁵ cells per dish. The following day, MSCGM was replaced with ODM or ODM containing BMP-7 at 500 ng/ml. After 24 hours of treatment, cells were trypsinized, fixed in ice cold 70% ethanol/30% PBS (v/v) for 30 minutes and centrifuged at 200×g. Pellets were resuspended in 800 μl PBS, 100 μL RNAse A (1 mg/ml) and 100 μL propidium iodide (400 μg/ml), incubated at 37° C. for exactly 30 minutes and placed on ice until analysis by flow cytometry. The flow cytometer was set to collect 10,000 events from each sample, and the cell cycle histograms were analyzed using ModFit LT (Verity Software House, N.Y.).

(i) Quantification of Cell Number

Primary hMSC were plated in MSCGM in 48-well dishes at 1.5×10⁴ cells per well. The following day, MSCGM was replaced with ODM or ODM containing BMP-7 at 500, 200 or 50 ng/ml. At the indicated time points, cell nuclei were stained with Hoechst 33342 (Invitrogen, Carlsbad, Calif.) and the number of nuclei in ten discrete fields was counted at 10× magnification using an ArrayScan VTI (Thermo Fisher, Waltham, Mass.). The accuracy of the cell counting algorithm employed was confirmed by generating a linear standard curve (R²=0.9975) from similarly stained cells seeded at fixed densities from 2.5×10³ to 80×10³ per well.

(j) Quantification of Cytokine Secretion

Tissue culture supernatants from hMSC treated with ODM or ODM containing 50 or 500 ng/ml BMP-7 were assayed for 30 human cytokines using a Human Cytokine 30-Plex Panel from Invitrogen (Carlsbad, Calif.). Cytokine analysis was performed by NovaScreen (Hanover, Md.). Briefly, beads conjugated to analyte-specific capture antibodies were incubated with cell supernatants or standard curve samples in 96-well plates. A biotinylated detector antibody was added to each well, followed by Streptavidin-RPE. Samples were analyzed in a Luminex 100 instrument. The concentration of each cytokine was determined from the standard curve, which was generated using a five parameter algorithm.

(k) Statistics for Follow-Up Studies

Data from time series experiments were analyzed by ANCOVA. Data from flow cytometry, cell cycle gene expression, cell quantification and siRNA knockdown studies were analyzed by two-sample t-tests and two-way ANOVA. Tukey HSD tests for multiple comparisons were used for all statistical analyses.

(l) Gene Ontology Trees

After categorization of significantly regulated genes in the data set into three major expression profiles, based on the temporal pattern and directionality of modulation by BMP-7 [Profiles A (A), B (B) and C (C)], directed acyclic graphs (GO Trees) were generated to depict the enriched gene ontology categories (categories with a hypergeometric test p-value <0.05 and at 2 least probe sets, which are colored red), and their non-enriched parents (which are colored black), for each profile.

(m) Comprehensive List of DEGs

Affymetrix® microarrays were used to evaluate BMP-7 mediated changes in global gene expression during early osteoblastic differentiation of primary hMSC.

Probes significantly (p≦0.01) regulated by at least two-fold with BMP-7 treatment, using both the MASS and RMA normalization strategies, were considered differentially expressed. 955 probe sets representing 655 known genes and 95 ESTs were identified at either 24 or 120 hours and grouped by hierarchical clustering into three major expression profiles as follows: up-regulated genes (Profile A) (A), transiently down-regulated genes (Profile B) (B), and continuously down-regulated genes (Profile C) (C).

(n) Complete KEGG Analysis

KEGG analysis was performed on differentially regulated genes from Profiles A (A), B (B) and C (C). Data were compiled and evaluated for depiction of KEGG pathways represented within each profile, the Entrez Gene IDs of the differentially regulated genes found within each pathway, and the extent of pathway enrichment. “O” is the observed gene number in the KEGG pathway, “E” is the expected number of genes in the KEGG pathway and “R” is the ratio of enrichment for the KEGG pathway (R=O/E). P-values for the significance of KEGG pathway enrichment were calculated using hypergeometric tests.

(o) Complete Gene Ontology Results

Gene ontology (GO) categories represented in the lists of differentially regulated genes were determined for Profiles A (A), B (B) and C (C). Data were compiled and evaluated for depiction of all GO categories (biological process, molecular function, cell component) for each differentially regulated probe set within a profile, along with the associated Entrez Gene ID, RefSeq ID, Gene Title and Gene Symbol for each probe set.

(p) Genes in Skeletal Development

Genes classified into the GO category of ‘Skeletal Development’ were evaluated in detail.

(q) Modulation of BMP, GDF, and inhibitor genes

Unfiltered RMA normalized data set was screened to generate comprehensive lists of BMP inhibitors and other BMP and GDF genes. Data was collected and evaluated relating to corresponding fold-changes, and ANOVA and t-test p-values in BMP-7 treated versus control cells. If the array contained multiple probes for a single gene, each probe is displayed individually.

### Example 2

### Overview of Gene Profiling During BMP-7 Mediated Osteoblastic Differentiation of Primary hMSC

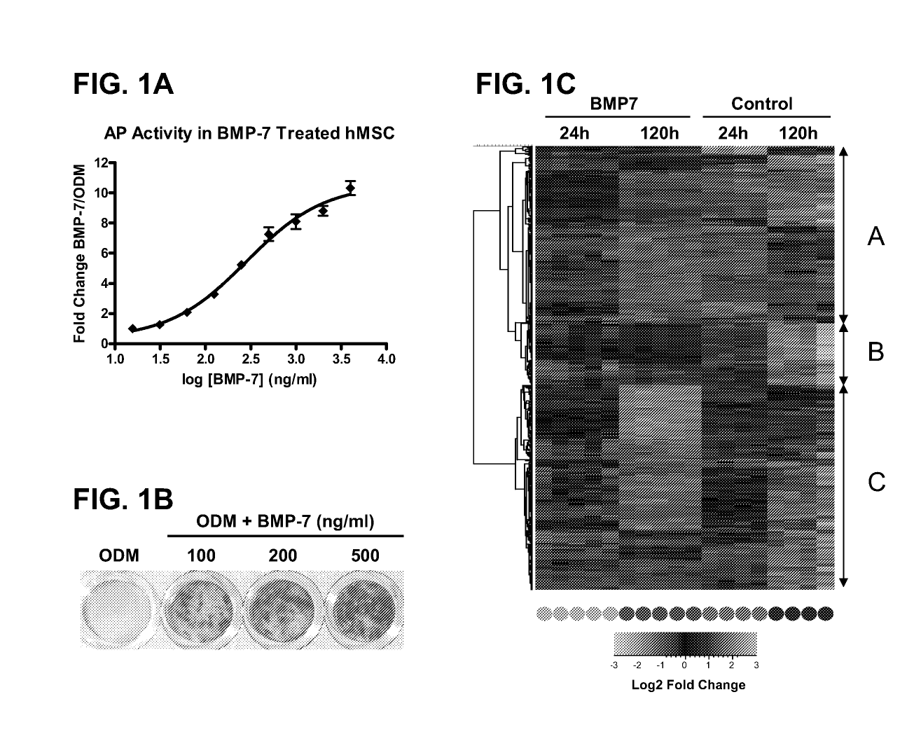

Affymetrix microarrays were used to evaluate BMP-7 mediated changes in global gene expression during early osteoblastic differentiation. The dose of BMP-7 used was established through preliminary work in which the response of primary hMSC in AP activity assays was assessed over a range of BMP-7 doses from 4 μg/ml to 16 ng/ml. Primary hMSC were seeded in 96-well dishes at 5×10³ cells per well and cultured in ODM alone or ODM supplemented with 1:2 serial dilutions of BMP-7 from 4 μg/ml to 16 ng/ml. AP activity was assessed after 6 days of treatment. The data are shown in FIG. 1A and are normalized to total protein and expressed as fold change in BMP-7 treated cells relative to ODM alone. Values shown represent the mean±S.D. of treatment wells. AP activity was dose-responsive with an approximate EC50 of 200-400 ng/ml BMP-7.

Likewise, in mineralization assays, hMSC demonstrated a dose-dependent increase in calcium deposition in response to BMP-7 treatment from 100-500 ng/ml. Primary hMSC were seeded in 48-well dishes at 1.5×10⁴ cells per well and cultured in ODM alone or ODM supplemented with 100, 200 or 500 ng/ml BMP-7. Cells were stained with Alizarin red after 17 days of treatment to assess mineralization. Data are shown in FIG. 1B. We therefore selected a dose of 500 ng/ml BMP-7 for our Affymetrix study, in order to induce robust changes in gene expression and minimize the possibility of missing biologically relevant effects.

For the Affymetrix study, primary hMSC were treated with ODM alone or ODM supplemented with 400 ng/ml of BMP-7 for 24 or 120 hours and processed on Affymetrix HG-U133 Plus 2.0 Arrays for analysis of gene expression over the entire human genome. Heat map depicts RMA-normalized signal values for probe sets with a 2-fold change and a p-value cut-off ≦0.01 for both MASS and RMA normalized data. Heat map coloration is based on log₂ signal values standardized by row mean centering. Significantly regulated genes were categorized into three major expression profiles based on the temporal pattern and directionality of modulation by BMP-7.

In order to focus our investigation on the principal downstream effects of BMP-7 treatment, and to avoid false positives, we imposed stringent criteria to generate lists of DEGs. Only probes significantly (p≦0.01) regulated by at least two-fold with BMP-7 treatment, using both the MASS and RMA normalization strategies, were considered differentially expressed. Applying these criteria, Affymetrix profiling identified 955 probe sets representing 655 known genes and 95 ESTs as differentially expressed at either 24 or 120 hours.

Hierarchical clustering of the 955 probe sets identified three major expression patterns, referred to as Profiles A, B and C which are shown in FIG. 1C.

Profile A contained genes that were up-regulated with BMP-7 treatment relative to controls. Profile B contained genes that were transiently down-regulated at 24 hours relative to controls and then became up-regulated by 120 hours, while Profile C contained genes that remained down-regulated with BMP-7 treatment for the duration of the five day study.

Certain of the most highly regulated genes within each profile are presented in Table 1. Full gene compilations were prepared as described elsewhere herein. The biological functions of the genes comprising the three profiles were then investigated using KEGG pathway analysis and GO classification. Complete compilations of the KEGG and GO analyses were prepared as described elsewhere herein. An abridged version of the KEGG results, showing the most significant and least redundant pathways, is presented in Table 2.

TABLE 1
Top Genes Regulated from Each Profile

| | | | | |
|---|---|---|---|---|
| | Fold Change | Fold Change | | |
| | BMP-7 versus | BMP-7 versus | Entrez | |
| Gene | Control | Control | Gene | |
| symbol | (24 hours) | (120 hours) | ID | Gene name |

A: Profile A

| | | | | |
|---|---|---|---|---|
| FGFR3 | 1.29-10.45 | 8.20-46.87 | 2261 | fibroblast growth factor |
| | | | | receptor 3 |
| ACTC | 4.79 | 41.57 | 70 | actin, alpha, cardiac muscle |
| HEY1 | 5.84-8.36 | 27.06-40.54 | 23462 | hairy/enhancer-of-split |
| | | | | related with YRPW motif 1 |
| DIO2 | 1.68-1.69 | 32.61-39.16 | 1734 | deiodinase, iodothyronine, |
| | | | | type II |
| NOG | 8.85 | 29.68 | 9241 | noggin |
| HAS3 | 4.30 | 20.81 | 3038 | hyaluronan synthase 3 |
| MCAM | 2.37-3.49 | 7.87-19.73 | 4162 | melanoma cell adhesion |
| | | | | molecule |
| ADAMTS9 | 1.16-1.70 | 7.93-19.16 | 56999 | a disintegrin-like and |
| | | | | metalloprotease with |
| | | | | thrombospondin type 1 |
| | | | | motif, 9 |
| MFI2 | 1.46 | 17.86 | 4241 | antigen p97 identified by |
| | | | | monoclonal antibodies |
| | | | | 133.2 and 96.5 |
| FRAS1 | 2.14 | 16.05 | 80144 | Fraser syndrome 1 |
| FAT3 | 0.93-0.97 | 2.82-15.71 | 120114 | FAT tumor suppressor |
| | | | | homolog 3 (Drosophila) |
| LOC440995 | 1.65 | 15.66 | 440995 | Hypothetical gene |
| | | | | supported by BC034933; |
| | | | | BC068085 |
| CD24 | 1.74-2.41 | 9.06-14.92 | 100133 | CD24 antigen (small cell |
| | | | 941 | lung carcinoma cluster 4 |
| | | | | antigen) |
| LMO2 | 4.66 | 14.71 | 4005 | LIM domain only 2 |
| | | | | (rhombotin-like 1) |
| AGC1 | 2.02-2.07 | 11.55-14.65 | 176 | aggrecan 1 (chondroitin |
| | | | | sulfate proteoglycan 1) |

B: Profile B

| | | | | |
|---|---|---|---|---|
| ANLN | 0.32-0.53 | 3.16-3.33 | 54443 | anillin, actin binding |
| | | | | protein (scraps homolog, |
| | | | | Drosophila) |
| CTSC | 0.34 | 1.62 | 1075 | cathepsin C |
| UHRF1 | 0.34 | 2.41 | 29128 | ubiquitin-like, containing |
| | | | | PHD and RING finger |
| | | | | domains, 1 |
| MAD2L1 | 0.38-0.49 | 2.47-3.03 | 4085 | MAD2 mitotic arrest |
| | | | | deficient-like 1 (yeast) |
| RRM2 | 0.40-0.53 | 2.82-4.03 | 6241 | ribonucleotide reductase |
| | | | | M2 polypeptide |
| MCM10 | 0.41-0.63 | 2.11-3.33 | 55388 | MCM10 minichromosome |
| | | | | maintenance deficient 10 |
| | | | | (S. cerevisiae) |
| HELLS | 0.41-0.46 | 2.46-3.27 | 3070 | Helicase, lymphoid- |
| | | | | specific |
| CCNE2 | 0.42 | 4.28 | 9134 | cyclin E2 |
| RAMP | 0.43-0.49 | 2.98-4.54 | 51514 | PvA-regulated nuclear |
| | | | | matrix-associated protein |
| TYMS | 0.44 | 2.12 | 7298 | thymidylate synthetase |
| DEPDC1 | 0.44-0.75 | 2.5-4.30 | 55635 | DEP domain containing 1 |
| KIF23 | 0.45 | 3.29 | 9493 | kinesin family member 23 |
| — | 0.45 | 1.84 | — | CDNA clone |
| | | | | IMAGE: 4452583, partial |
| | | | | cds |
| KIF11 | 0.46 | 3.39 | 3832 | kinesin family member 11 |
| ZNF367 | 0.47 | 3.36 | 195828 | zinc finger protein 367 |

C: Profile C

| | | | | |
|---|---|---|---|---|
| CHI3L1 | 0.54-0.62 | 0.00-0.01 | 1116 | chitinase 3-like 1 (cartilage |
| | | | | glycoprotein-39) |
| WISP2 | 0.88 | 0.03 | 8839 | WNT1 inducible signaling |
| | | | | pathway protein 2 |
| SFRP4 | 1.17-1.50 | 0.05-0.07 | 6424 | secreted frizzled-related |
| | | | | protein 4 |
| CCL2 | 0.24 | 0.06 | 6347 | chemokine (C-C motif) |
| | | | | ligand 2 |
| EVI2A | 0.52 | 0.06 | 2123 | ecotropic viral integration |
| | | | | site 2A |
| CH25H | 0.52 | 0.08 | 9023 | cholesterol 25-hydroxylase |
| GDF15 | 1.50 | 0.08 | 9518 | growth differentiation |
| | | | | factor 15 |
| IL6 | 0.48 | 0.09 | 3569 | interleukin 6 (interferon, |
| | | | | beta 2) |
| EGR3 | 0.54 | 0.09 | 1960 | early growth response 3 |
| ADM | 0.89 | 0.09 | 133 | adrenomedullin |
| DDIT4L | 0.42 | 0.09 | 115265 | DNA-damage-inducible |
| | | | | transcript 4-like |
| FLG | 0.45 | 0.10 | 2312 | filaggrin (previously |
| | | | | hypothetical gene |
| | | | | supported by M60502) |
| SLC2A5 | 0.52 | 0.12 | 6518 | solute carrier family 2 |
| | | | | (facilitated |
| | | | | glucose/fructose |
| | | | | transporter), member 5 |
| MEST | 0.44 | 0.13 | 4232 | mesoderm specific |
| | | | | transcript homolog |
| | | | | (mouse) |
| DCAMKL1 | 0.65-0.67 | 0.13-0.22 | 9201 | doublecortin and CaM |
| | | | | kinase-like 1 |

RMA-normalized signal values were used to generate tables of the 15 most regulated genes in each profile by fold up-regulation at 120 hours (Profile A) (A), fold down-regulation at 24 hours (Profile B) (B), fold down-regulation at 120 hours (Profile C) (C). A range of fold change values is presented for genes represented by multiple probes on the Affymetrix microarrays.

TABLE 2
KEGG Analysis

| | | | | | |
|---|---|---|---|---|---|
| | Fold Change | Fold Change | | | |
| | BMP-7 versus | BMP-7 versus | Entrez | | |
| Pathway/ | Control | Control | Gene | | |
| Gene symbol | (24 hours) | (120 hours) | ID | Gene name | p-value |

A: Profile A

| | | | | | |
|---|---|---|---|---|---|
| TGF-beta | | | | | P = 8.03e−5 |
| signaling | | | | | |
| pathway | | | | | |
| COMP | 4.80 | 4.60 | 1311 | cartilage oligomeric matrix | |
| | | | | protein | |
| ID3 | 1.85 | 2.66 | 3399 | inhibitor of DNA binding | |
| | | | | 3, dominant negative helix- | |
| | | | | loop-helix protein | |
| ID4 | 1.25-1.77 | 2.45-2.57 | 3400 | inhibitor of DNA binding | |
| | | | | 4, dominant negative helix- | |
| | | | | loop-helix protein | |
| INHBA | 1.29-1.52 | 2.09-2.38 | 3624 | inhibin, beta A | |
| SMAD6 | 1.99 | 3.59 | 4091 | SMAD family member 6 | |
| SMAD7 | 1.96 | 2.84 | 4092 | SMAD family member 7 | |
| NOG | 8.85 | 29.68 | 9241 | noggin | |
| Axon | | | | | p = 4.51e−3 |
| guidance | | | | | |
| UNC5B | 2.5-2.64 | 3.81-3.93 | 219699 | unc-5 homolog B (C. | |
| | | | | elegans) | |
| GNAI1 | 1.29-1.45 | 2.14-2.19 | 2770 | guanine nucleotide binding | |
| | | | | protein (G protein), alpha | |
| | | | | inhibiting activity | |
| | | | | polypeptide 1 | |
| LIMK2 | 1.73 | 2.46 | 3985 | LIM domain kinase 2 | |
| PLXNA2 | 1.84 | 3.40 | 5362 | plexin A2 | |
| RGS3 | 1.82 | 3.57 | 5998 | regulator of G-protein | |
| | | | | signaling 3 | |
| SEMA6D | 1.96-3.24 | 2.91-4.29 | 80031 | sema domain, | |
| | | | | transmembrane domain | |
| | | | | (TM), and cytoplasmic | |
| | | | | domain, (semaphorin) 6D | |
| Wnt | | | | | p = 8.91e−3 |
| signaling | | | | | |
| pathway | | | | | |
| DKK1 | 1.77 | 2.78 | 22943 | dickkopf homolog 1 | |
| | | | | (Xenopus laevis) | |
| LEF1 | 1.15 | 2.57 | 51176 | LEF1, lymphoid enhancer- | |
| | | | | binding factor 1 | |
| SIAH1 | 1.14 | 2.06 | 6477 | seven in absentia homolog | |
| | | | | 1 (Drosophila) | |
| WNT2B | 1.07 | 2.88 | 7482 | wingless-type MMTV | |
| | | | | integration site family, | |
| | | | | member 2B | |
| FZD1 | 1.48 | 2.11 | 8321 | 8321 = FZD1, frizzled | |
| | | | | homolog 1 | |
| FZD8 | 2.65-3.40 | 4.43-6.38 | 8325 | 8325 = FZD8, frizzled | |
| | | | | homolog 8 | |
| Jak-STAT | | | | | p = 9.24e−3 |
| signaling | | | | | |
| pathway | | | | | |
| GHR | 1.78 | 3.27 | 2690 | growth hormone receptor | |
| IL7R | 1.45-1.69 | 3.98-7.29 | 3575 | interleukin 7 receptor | |
| LIFR | 0.87 | 3.34 | 3977 | leukemia inhibitory factor | |
| | | | | receptor alpha | |
| STAT4 | 1.49 | 2.27 | 6775 | signal transducer and | |
| | | | | activator of transcription 4 | |
| PIK3R3 | 0.92 | 2.01 | 8503 | PIK3R3, phosphoinositide- | |
| | | | | 3-kinase | |
| SOCS2 | 1.85-1.37 | 4.40-6.61 | 8835 | suppressor of cytokine | |
| | | | | signaling 2 | |
| Focal | | | | | p = 1.12e−2 |
| adhesion | | | | | |
| COMP | 4.80 | 4.60 | 1311 | cartilage oligomeric matrix | |
| | | | | protein | |
| FLNB | 1.30 | 2.36 | 2317 | filamin B, beta (actin | |
| | | | | binding protein 278) | |
| IGF1 | 1.11-2.52 | 2.91-12.00 | 3479 | insulin-like growth factor 1 | |
| | | | | (somatomedin C) | |
| ITGA9 | 1.66 | 12.09 | 3680 | integrin, alpha 9 | |
| PGF | 8.54 | 11.40 | 5228 | placental growth factor | |
| ACTC1 | 4.79 | 41.57 | 70 | actin, alpha, cardiac muscle | |
| | | | | 1 | |
| PIK3R3 | 0.92 | 2.01 | 8503 | phosphoinositide-3-kinase | |
| Regulation | | | | | p = 3.89e−2 |
| of actin | | | | | |
| cytoskele- | | | | | |
| ton | | | | | |
| F2R | 1.02 | 2.41 | 2149 | coagulation factor II | |
| | | | | (thrombin) receptor | |
| FGFR3 | 1.29-10.45 | 8.20-46.87 | 2261 | fibroblast growth factor | |
| | | | | receptor 3 (achondroplasia, | |
| | | | | thanatophoric dwarfism) | |
| ITGA9 | 1.66 | 12.09 | 3680 | integrin, alpha 9 | |
| LIMK2 | 1.73 | 2.46 | 3985 | LIM domain kinase 2 | |
| ACTC1 | 4.79 | 41.57 | 70 | actin, alpha, cardiac muscle | |
| | | | | 1 | |
| PIK3R3 | 0.92 | 2.01 | 8503 | phosphoinositide-3-kinase | |

B: Profile B

| | | | | | |
|---|---|---|---|---|---|
| Cell cycle | | | | | P = 8.30e−9 |
| MAD2L1 | 0.38-0.49 | 2.47-3.03 | 4085 | MAD2 mitotic arrest | |
| | | | | deficient-like 1 (yeast) | |
| | | | | minichromosome | |
| MCM4 | 0.73 | 2.38 | 4173 | maintenance complex | |
| | | | | component 4 | |
| RBL1 | 0.59 | 2.37 | 5933 | retinoblastoma-like 1 | |
| | | | | (p107) | |
| CCNA2 | 0.63-0.79 | 2.34-2.61 | 890 | cyclin A2 | |
| CCNB1 | 0.55-0.66 | 2.52-3.78 | 891 | cyclin B1 | |
| CCNB2 | 0.69 | 2.40 | 9133 | cyclin B2 | |
| CCNE2 | 0.42 | 4.28 | 9134 | cyclin E2 | |
| CDC2 | 0.50-0.61 | 3.11-3.68 | 983 | cell division cycle 2, | |
| | | | | G1 to S and G2 to M | |
| CDC6 | 0.49-0.62 | 2.33-3.32 | 990 | cell division cycle 6 | |
| | | | | homolog (S. cerevisiae) | |
| Pyrimidine | | | | | P = 1.49e−3 |
| metabolism | | | | | |
| CTPS | 0.78 | 2.10 | 1503 | CTP synthase | |
| POLE2 | 0.68 | 2.13 | 5427 | polymerase (DNA | |
| | | | | directed), epsilon 2 | |
| | | | | (p59 subunit) | |
| RRM2 | 0.40-0.53 | 2.82-4.03 | 6241 | ribonucleotide | |
| | | | | reductase M2 | |
| | | | | polypeptide | |
| TYMS | 0.44 | 2.12 | 7298 | thymidylate synthetase | |

C: Profile C

| | | | | | |
|---|---|---|---|---|---|
| Cytokine- | | | | | P = 2.68e−3 |
| cytokine | | | | | |
| receptor | | | | | |
| interaction | | | | | |
| TNFSF13B | 1.27 | 0.44 | 10673 | tumor necrosis factor | |
| | | | | (ligand) superfamily, | |
| | | | | member 13b | |
| IL6 | 0.48 | 0.09 | 3569 | interleukin 6 | |
| | | | | (interferon, beta 2) | |
| TNFRSF11B | 0.34-0.35 | 0.21-0.23 | 4982 | tumor necrosis factor | |
| | | | | receptor superfamily, | |
| | | | | member 11b | |
| | | | | (osteoprotegerin) | |
| CCL2 | 0.24 | 0.06 | 6347 | chemokine (C-C motif) | |
| | | | | ligand 2 | |
| CXCL12 | 0.79-0.93 | 0.36-0.46 | 6387 | chemokine (C-X-C | |
| | | | | motif) ligand 12 | |
| | | | | (stromal cell-derived | |
| | | | | factor 1) | |
| VEGFA | 0.62 | 0.35 | 7422 | vascular endothelial | |
| | | | | growth factor A | |
| VEGFB | 0.98 | 0.45 | 7423 | vascular endothelial | |
| | | | | growth factor B | |
| GDF5 | 0.38 | 0.31 | 8200 | growth differentiation | |
| | | | | factor 5 | |
| TNFSF10 | 0.82-0.93 | 0.20-0.46 | 8743 | tumor necrosis factor | |
| | | | | (ligand) superfamily, | |
| | | | | member 10 | |
| Purine | | | | | P = 1.98e−2 |
| metabolism | | | | | |
| ADA | 1.30 | 0.43 | 100 | adenosine deaminase | |
| AK5 | 0.77-0.91 | 0.42-0.50 | 26289 | adenylate kinase 5 | |
| NT5E | 0.68-0.90 | 0.38-0.48 | 4907 | 5′-nucleotidase, ecto | |
| | | | | (CD73) | |
| PDE1A | 0.99 | 0.37 | 5136 | phosphodiesterase 1A, | |
| | | | | calmodulin-dependent | |
| ENPP1 | 0.66 | 0.33 | 5167 | ectonucleotide | |
| | | | | pyrophosphatase/phos- | |
| | | | | phodiesterase 1 | |
| Nicotinate | | | | | 2.74E−04 |
| and | | | | | |
| nicotinamide | | | | | |
| metabolism | | | | | |
| NAMPT | 0.89-0.92 | 0.32-0.37 | 10135 | nicotinamide | |
| | | | | phosphoribosyltransfer- | |
| | | | | ase (PBEF1) | |
| AOX1 | 0.92 | 0.20 | 316 | aldehyde oxidase 1 | |
| NNMT | 0.44 | 0.40 | 4837 | nicotinamide N- | |
| | | | | methyltransferase | |
| NT5E | 0.68-0.90 | 0.38-0.48 | 4907 | 5′-nucleotidase, ecto | |
| | | | | (CD73) | |
| | | | | ectonucleotide | |
| ENPP1 | 0.66 | 0.33 | 5167 | pyrophosphatase/phos- | |
| | | | | phodiesterase 1 | |
| ECM- | | | | | P = 3.23e−2 |
| receptor | | | | | |
| interaction | | | | | |
| FN1 | 1.15 | 0.47 | 2335 | fibronectin 1 | |
| LAMA1 | 0.80 | 0.37 | 284217 | laminin, alpha 1 | |
| SDC4 | 0.54 | 0.28 | 6385 | syndecan 4 | |
| CD44 | 0.72-0.90 | 0.41-0.45 | 960 | CD44 molecule (Indian | |
| | | | | blood group) | |
| Complement | | | | | P = 1.85e−2 |
| and | | | | | |
| coagulation | | | | | |
| cascades | | | | | |
| SERPINE1 | 0.35-0.44 | 0.18-0.37 | 5054 | serpin peptidase | |
| | | | | inhibitor, clade E | |
| | | | | (nexin, plasminogen | |
| | | | | activator inhibitor type | |
| | | | | 1), member 1 | |
| PLAU | 0.82-1.02 | 0.34-0.38 | 5328 | plasminogen activator, | |
| | | | | urokinase | |
| BDKRB2 | 1.01 | 0.46 | 624 | bradykinin receptor B2 | |
| CFB | 1.18 | 0.21 | 629 | complement factor B | |
| PPAR | | | | | P = 1.85e−2 |
| signaling | | | | | |
| pathway | | | | | |
| FABP3 | 1.05 | 0.48 | 2170 | fatty acid binding | |
| | | | | protein 3, muscle and | |
| | | | | heart (mammary- | |
| | | | | derived growth | |
| | | | | inhibitor) | |
| ACSL1 | 0.86 | 0.45 | 2180 | acyl-CoA synthetase | |
| | | | | long-chain family | |
| | | | | member 1 | |
| PLTP | 1.31 | 0.47 | 5360 | phospholipid transfer | |
| | | | | protein | |
| SCD | 1.45-1.86 | 0.28-0.30 | 6319 | stearoyl-CoA | |
| | | | | desaturase (delta-9- | |
| | | | | desaturase) | |

KEGG analysis was performed on differentially regulated genes from Profiles A (A), B (B) and C (C). Tables depict the most significant and least redundant KEGG pathways enriched within each profile, along with associated hypergeometric test p-values. If the array contained multiple probes for a single gene, the range of detection is reported.

### Example 3

### BMP-7 Up-Regulates Markers of Osteoblastic Differentiation

Consistent with a model of BMP bioactivity, a strong up-regulation of genes associated with TGF-beta signaling was revealed through KEGG analysis of Profile A (Table 2A). Genes within this category include the BMP-responsive transcriptional regulators ID3 and ID4, and the BMP inhibitors NOG, SMAD6 and SMAD7. Also observed within Profile A were a number of recognized markers of BMP mediated osteoblastic differentiation, including the non-specific osteoblast markers ALPL and PGF (Marrony et al., (2003), Bone, 33:426-33). Members of the distal-less homeobox (dlx) family, including DLX1, DLX2, DLX3, DLX5 and DLX6, and the msh homeobox homolog (msx) family, including MSX1 and MSX2, were prominent in Profile A. These transcriptional regulators are directly induced by BMPs and control the expression of later-stage transcription factors (Ryoo et al., (2006), Gene, 366:51-7). Significant up-regulation (>36-fold) of SP7 (osterix), which coordinates the expression of downstream osteoblast-associated genes, was also observed (Kim et al., (2006), Gene, 366:145-51). Many of the osteoblast-associated genes were categorized into the functional classification of ‘Skeletal Development’ by GO analysis as indicated elsewhere herein. The identification of these genes among those regulated by BMP-7 confirms that MSC analyzed by Affymetrix profiling in this experiment had initiated a normal BMP mediated osteoblastic differentiation.

### Example 4

### BMP-7 Strongly Up-Regulates Genes with Previously Undefined Roles in Osteoblastic Differentiation

Among the most highly up-regulated genes in Profile A were several genes with previously unknown or poorly defined roles in BMP mediated osteoblastic differentiation. These include but are not limited to FGFR3, DIO2, HEY1, HAS3, ADAMTS9 and MFI2 (See Table 1A). The trends in gene expression suggested in the Affymetrix profiling were confirmed by QPCR in subsequent experiments.

Primary hMSC were seeded in 48-well dishes at 1.5×10⁴ cells per well and cultured in ODM alone or ODM supplemented with 40 or 400 ng/ml BMP-7. Cells were lysed after 1, 2, 4 or 7 days of treatment. Expression of FGFR3, DIO2, HEY1, ADAMTS9, HAS3 and MFI2 mRNA was quantified by RT-QPCR and normalized to GAPDH. The data is presented in FIGS. 2A-F. The values shown representing the mean±S.D. of triplicate measurements and are expressed relative to treatment with ODM alone at day one. BMP-7 treatments that are significantly different (p<0.05) from the control are indicated by dotted lines.

Of those genes which showed a dose-responsive increase in expression in BMP-7 treated hMSC over seven days, the following exemplary genes showed a maximum up-regulation of approximately: 500-fold (FGFR3), 490-fold (DIO2), 250-fold (HEY1), 160-fold (ADAMTS9), 110-fold (HAS3) and 40-fold (MFI2). Data were significantly different from ODM controls at both 40 ng/ml and 400 ng/ml BMP-7 for all six genes (p<0.05).

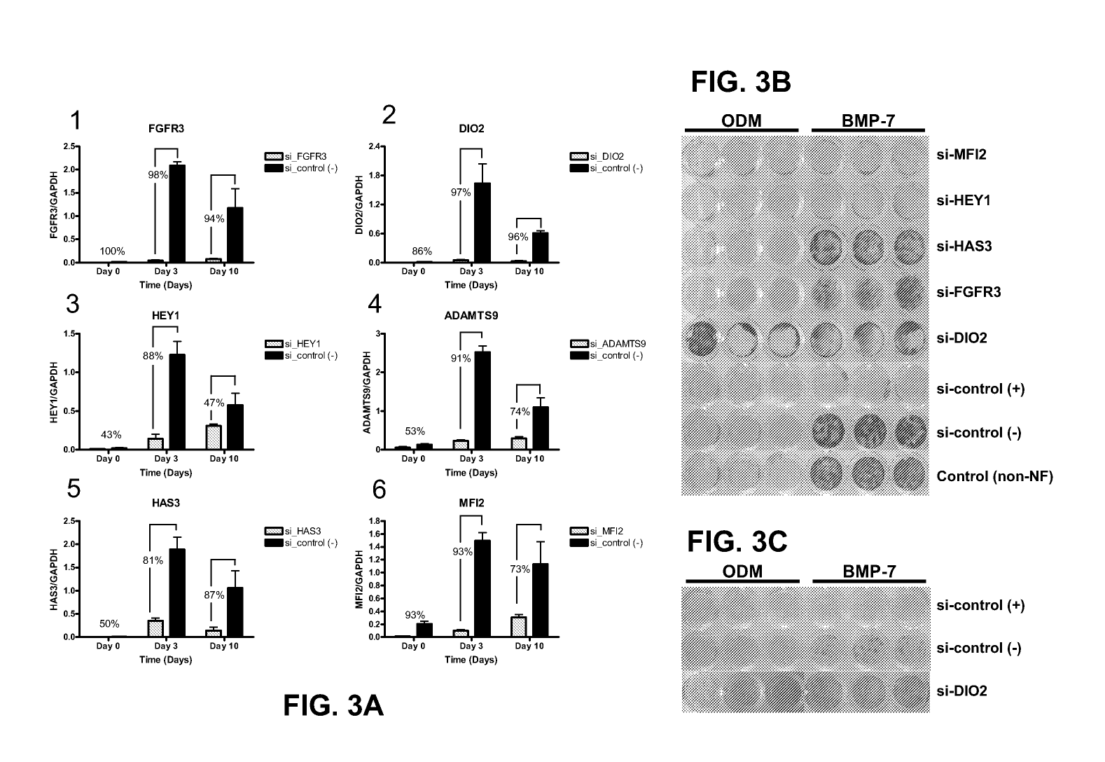

We then evaluated the contribution of some of these same genes to BMP-7 mediated osteoblastic differentiation. Using siRNA, expression of a target gene was inhibited, and the ability of MSC to differentiate into mineralizing osteoblasts following BMP-7 treatment was assessed. Primary hMSC were nucleoporated with a total of 4 μg siRNA targeting FGFR3, DIO2, HEY1, ADAMTS9, HAS3 or MFI2 and seeded into 48-well dishes at 1.5×10⁴ cells per well in MSCGM. Positive and negative control nucleoporations were performed as described in Materials and Methods. 48 hours after nucleoporation, cells were treated with ODM alone or ODM containing 200 ng/ml of BMP-7. Cells were harvested at Day 0, 3 or 10 after BMP-7 treatment (2, 5 and 12 days after nucleofection, respectively) to assess target gene knockdown by RT-QPCR. Data is shown in FIG. 3A. Cells were stained with Alizarin red after 12 days of BMP-7 treatment, to assess mineralization as shown in FIG. 3B. Cells nucleofected with siRNA targeting DIO2 were stained with Alizarin red after 9 days of nucleofection showed advanced mineralization prior to detachment as shown in FIG. 3C.

Potent and persistent down-regulation of each target gene was documented at several time points through Day 10 of BMP-7 treatment (12 days after nucleoporation) (FIG. 3A). Mineralization was assessed by Alizarin Red staining after 12 days of BMP-7 treatment (FIG. 3B). As expected, robust mineralization of BMP-7 treated hMSC was observed in cells transfected with non-targeted siRNA and in non-nucleoporated cells, whereas no mineralization was detected in cells nucleoporated with pooled siRNA targeting BMPR1A and ACVR1. Knockdown of MFI2 and HEY1 completely blocked the mineralizing capacity of the cells (FIG. 3B). In contrast, knockdown of DIO2 unexpectedly led to an accelerated mineralization process in both BMP-7 and control (ODM) treated cells. Significant mineral deposition was apparent as early as day 9 post treatment (FIG. 3C). After this time, cells began to detach from the plates. By day 12, very few cells remained, although mineral deposited by the cells was still detectable (FIG. 3B). Nucleofection of hMSC, for example, with siRNA targeting HAS3 and FGFR3 did not significantly affect mineralization (FIG. 3B).

### Example 5

### BMP-7 Induces an Unexpected Transient Down-Regulation of the Cell Cycle During Early Osteoblastic Differentiation

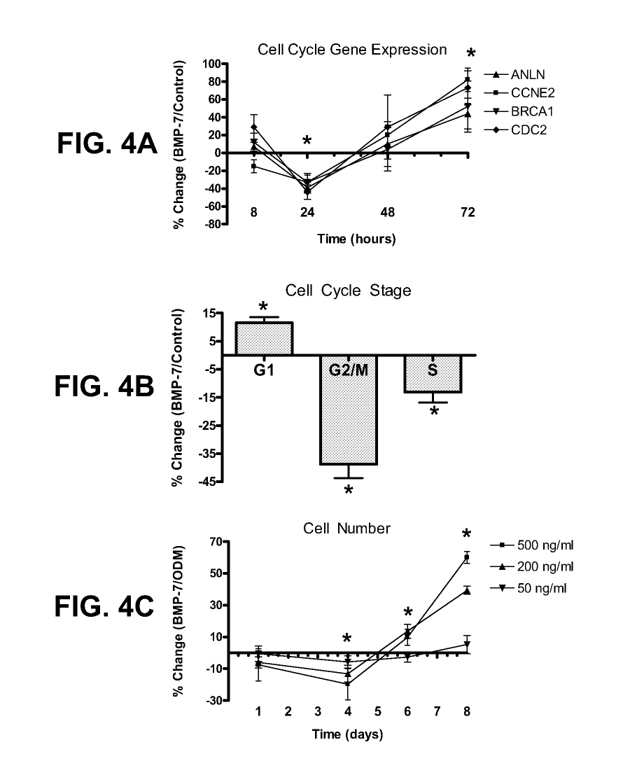

Enrichment for genes associated with cell cycle and DNA replication was identified in Profile B (transiently down-regulated at 24 hours, then up-regulated at 120 hours by BMP-7 treatment) by KEGG analysis (See Table 2B). Further investigation revealed that additional cell cycle-associated genes, classified into Profile B but not identified by KEGG analysis, were similarly regulated (See Table 3 below). Modulation of expression of a subset of cell cycle-associated genes, including CCNE2, ANLN, CDC2, and BRCA1, was confirmed by QPCR in follow-up studies. Accordingly, primary hMSC were seeded in 48-well dishes at 1.5×10⁴ cells per well and cultured in ODM alone or ODM supplemented with 400 ng/ml BMP-7. Cells were lysed after 8, 24, 48 or 72 hours of treatment. Expression of CCNE2, ANLN, BRCA1 and CDC2 mRNA was quantified by RT-QPCR and normalized to GAPDH. The data is shown in FIG. 4A. All four genes were significantly (p<0.05) down-regulated in parallel by approximately 40% at 24 hours by BMP-7 treatment, and became significantly (p<0.05) up-regulated by 40-80% within three days of BMP-7 treatment, confirming the trend in gene expression observed in the Affymetrix study.

Since the magnitude of the observed gene down-regulation at 24 hours was relatively minor, and an inhibitory role for BMPs on cell cycle progression is not widely recognized, the early effect of BMP-7 on cell cycle stage was evaluated using flow cytometry. Primary hMSC were seeded in 35-mm dishes at 1.5×10⁵ cells per dish and cultured in ODM alone or ODM supplemented with 500 ng/ml BMP-7. After 24 hours of treatment, cells were analyzed by flow cytometry as described above.

BMP-7 induced a statistically significant increase of approximately 12% (from 62.1% to 69.2% of total cell population, p=0.0021) in the percentage of cells in G0/G1 after 24 hours, with a simultaneous 39% and 13% decrease, respectively, in the number of cells in S phase (from 29.4% to 25.5%, p=0.0008) and G2/M (from 8.6% to 5.3%, p=0.01) as shown in FIG. 4B. The observed shifts were repeatable at both low and high seeding densities of MSC, verifying that the unexpected effect of cell cycle arrest was attributable to BMP-7 treatment and not contact inhibition associated with cell confluence.

The effect of BMP-7 on cell proliferation was further evaluated by quantifying the number of cells in BMP-7 treated hMSC over a period of 8 days as shown in FIG. 4C. Primary hMSC were seeded in 48-well dishes at 1.5×10⁴ cells per well and cultured in ODM alone or ODM supplemented with 500, 200 or 50 ng/ml BMP-7. At the indicated time points, cells were stained with Hoechst 33342 and the number of nuclei quantified. At the one and four day time points, a decrease in cell number relative to controls was observed. In contrast, an increase in cell number relative to controls was measured at days 6 and 8. These changes were statistically significant at 500 ng/ml BMP-7 at Day 4, 200 ng/ml BMP-7 at Day 6, and both 200 and 500 ng/ml BMP-7 at Day 8. Treatment of cells with 50 ng/ml of BMP-7 resulted in a similar trend to what was observed at higher doses, although the changes did not reach statistical significance.

Overall, these data are consistent with BMP-7 induction of cell cycle attenuation during early osteoblastic differentiation of hMSC, whereas BMP-7 has a measurable mitogenic effect at later stages of osteoblastic differentiation.

TABLE 3
Cell cycle-associated DEGs.
Cell cycle genes

| | Fold Change | Fold Change | | |
|---|---|---|---|---|
| | BMP-7 versus | BMP-7 versus | Entrez | |
| Gene | Control | Control | Gene | |
| symbol | (24 hours) | (120 hours) | ID | Gene name |
| ANLN | 0.32-0.53 | 3.16-3.33 | 54443 | anillin, actin binding protein |
| AURKB | 0.6 | 2.12 | 9212 | aurora kinase B |
| BIRC5 | 0.50-0.63 | 1.89-2.47 | 332 | baculoviral IAP repeat- |
| | | | | containing 5 (survivin) |
| BRCA1 | 0.65 | 2.29 | 672 | breast cancer 1, early onset |
| CCNB1 | 0.62-0.66 | 2.33-3.78 | 891 | cyclin B1 |
| CCNB2 | 0.69 | 2.4 | 9133 | cyclin B2 |
| CCNE2 | 0.42 | 4.28 | 9134 | cyclin E2 |
| CDC2 | 0.50-0.61 | 3.11-3.68 | 983 | cell division cycle 2, G1 to S |
| | | | | and G2 to M |
| CDC6 | 0.49-0.62 | 2.33-3.32 | 990 | CDC6 cell division cycle 6 |
| | | | | homo log (S. cerevisiae) |
| CDKN3 | 0.47-0.49 | 1.96-2.06 | 1033 | cyclin-dependent kinase |
| | | | | inhibitor 3 |
| DLG7 | 0.52 | 2.96 | 9787 | discs, large homolog 7 |
| | | | | (Drosophila) |
| GTSE1 | 0.64 | 3.11 | 51512 | G-2 and S-phase expressed 1 |
| KIF11 | 0.46 | 3.39 | 3832 | kinesin family member 11 |
| KIF23 | 0.45 | 3.29 | 9493 | kinesin family member 23 |
| KIF2C | 0.61 | 2.08 | 11004 | kinesin family member 2C |
| MAD2L1 | 0.38-0.49 | 2.47-3.03 | 4085 | MAD2 mitotic arrest deficient- |
| | | | | like 1 (yeast) |
| MCM8 | 0.68 | 2.61 | 84515 | MCM8 minichromosome |
| | | | | maintenance deficient 8 (S. |
| | | | | cerevisiae) |
| NEK2 | 0.59 | 3.09 | 4751 | NIMA (never in mitosis gene a)- |
| | | | | related kinase 2 |
| PLK4 | 0.53 | 2.06 | 10733 | polo-like kinase 4 (Drosophila) |
| PRC1 | 0.68 | 2.88 | 9055 | protein regulator of cytokinesis |
| | | | | 1 |
| RBL1 | 0.59 | 2.37 | 5933 | retinoblastoma-like 1 (p107) |
| SGOL2 | 0.71 | 2.8 | 151246 | shugoshin-like 2 (S. pombe) |
| SPAG5 | 0.59 | 2.33 | 10615 | sperm associated antigen 5 |
| TPX2 | 0.59 | 2.44 | 22974 | TPX2, microtubule-associated, |
| | | | | homolog (Xenopus laevis) |
| TTK | 0.65 | 2.92 | 7272 | TTK protein kinase |
| TUBG1 | 0.75 | 2.03 | 7283 | tubulin, gamma 1 |
| UBE2C | 0.54 | 2.87 | 11065 | ubiquitin-conjugating enzyme |
| | | | | E2C |
| ZWINT | 0.67 | 2.87 | 11130 | ZW10 interactor |

RMA-normalized signal values were used to generate a table of cell cycle-associated genes categorized into Profile B. A range of fold change values is presented for genes represented by multiple probes on the Affymetrix microarrays.

### Example 6

### BMP-7 Induces a Down-Regulation of Cytokine Expression

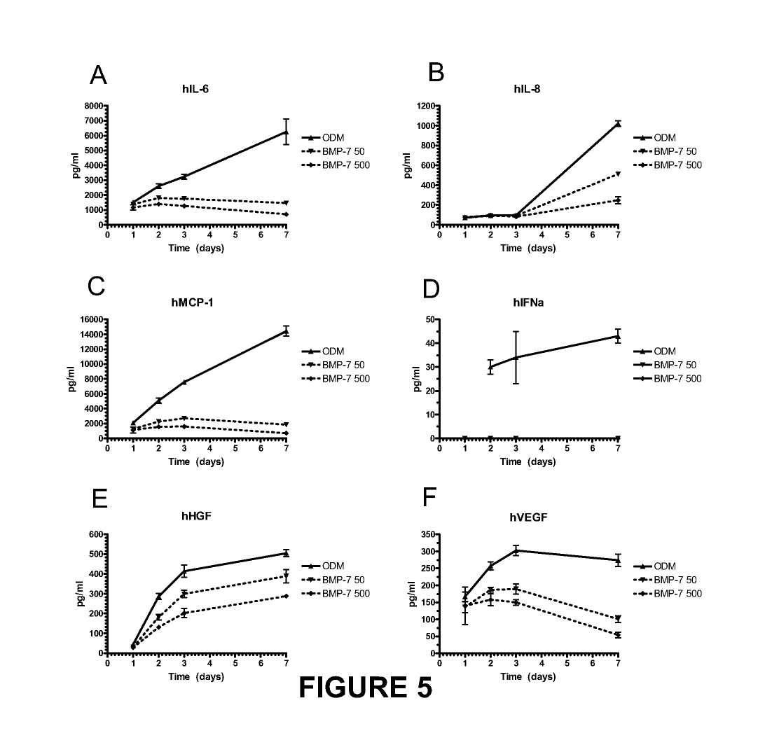

The most prominent pathway identified by KEGG analysis of Profile C (genes continuously down-regulated by BMP-7 treatment) was the cytokine-cytokine receptor interaction pathway (See Table 2C). Nine genes classified into this pathway were significantly down-regulated by BMP-7 treatment in the Affymetrix study. To confirm cytokine mRNA down-regulation at the secreted protein level, we performed an analysis of secreted cytokines in supernatants from BMP-7 treated hMSC with data presented in FIG. 5.

Primary hMSC were seeded in 24-well dishes at 3×10⁴ cells per well and cultured in ODM alone or ODM supplemented with 50 or 500 ng/ml BMP-7. Tissue culture supernatants were collected after 1, 2, 3 or 7 days of treatment and assayed using the Human Cytokine 30-plex Bead Immunoassay kit as described above. Thirty cytokines were assayed and detected at quantifiable levels, included among those were exemplary cytokines such as but not limited to IL6, IL8, MCP-1, IFNa, HGF and VEGF. Of these, IL6, IL8, MCP-1, HGF and VEGF, which were all down-regulated by BMP-7 treatment relative to ODM alone, in a dose-dependent manner. IFNa was detected in control cells, but not in cells treated with BMP-7 at either concentration. Data were significantly different from ODM controls at both 50 ng/ml and 500 ng/ml BMP-7 for all six proteins (p<0.05).

### Example 7

### BMP-7 Down-Regulates Cartilage Glycoprotein-39

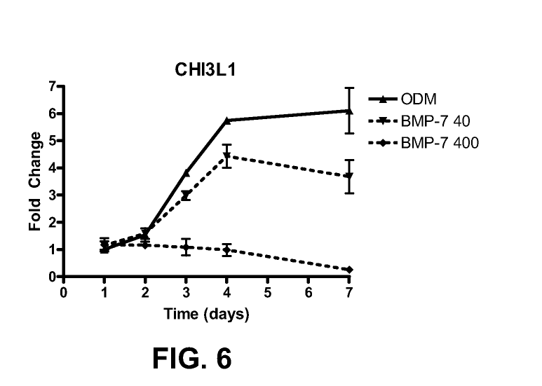

Notable among the genes inhibited by BMP-7 treatment in Profile C (Table 1C) was CHI3L1 (chitinase 3-like 1/cartilage glycoprotein-39/YKL-40). Two distinct probes on the Affymetrix array detected robust down-regulation of CHI3L1 of approximately 97% (probe 209395_at) and 93% (probe 209396_s_at). This effect of BMP-7 was heretofore unreported. Since the magnitude of down-regulation for CHI3L1 far exceeded that of all other genes in the analysis, modulation of expression was confirmed by QPCR in a follow-up experiment as shown in FIG. 6. Primary hMSC were seeded in 48-well dishes at 1.5×10⁴ cells per well and cultured in ODM alone or ODM supplemented with 40 or 400 ng/ml BMP-7. Cells were lysed after 1, 2, 3, 4 or 7 days of treatment. Expression of CHI3L1 mRNA was quantified by RT-QPCR and normalized to GAPDH. CHI3L1 was continuously down-regulated in a dose-dependent manner in hMSC treated with BMP-7 for seven days, with a maximum 24-fold down-regulation observed at the higher dose of BMP-7 relative to untreated cells. Data were significantly different from ODM controls at both 40 ng/ml and 400 ng/ml BMP-7 (p<0.05).

### Example 8

### BMP-7 Alone can Modulate Expression of BMP-2 and GDF5, and does Not Require Endogenous BMP-2 Expression to Induce Osteoblastic Differentiation

We interrogated the Affymetrix data to identify all BMP-7 mediated effects on the expression of BMPs, GDFs and known BMP inhibitors as described elsewhere herein. No thresholds for gene expression intensity or fold-change were imposed during this data acquisition. Every gene demonstrating a statistically significant modulation of ≧20% in BMP-7 versus control treated cells was further investigated by QPCR in confirmatory studies.

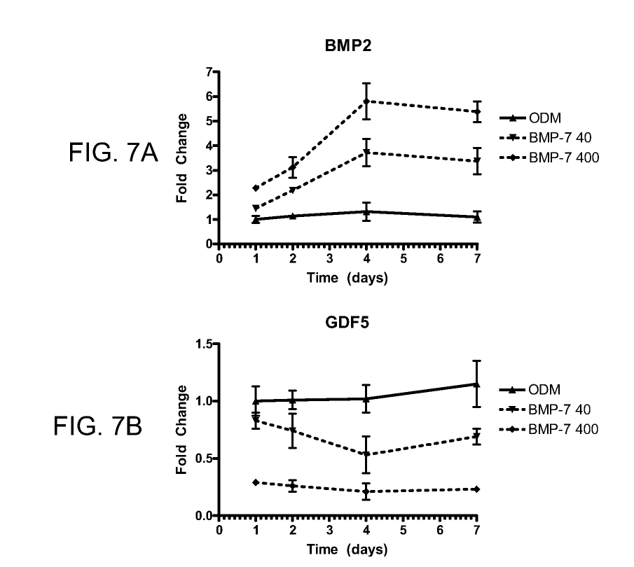

Of all BMP and GDF genes, only BMP-2 and GDF-5 analyses confirmed the trends suggested by the Affymetrix profiling. Primary hMSC were seeded in 48-well dishes at 1.5×10⁴ cells per well and cultured in ODM alone or ODM supplemented with 40 or 400 ng/ml BMP-7. Cells were lysed after 1, 2, 4 or 7 days of treatment. Expression of BMP-2 and GDF5 was quantified by RT-QPCR and normalized to GAPDH. As shown in FIGS. 7A-B, BMP-2 mRNA expression increased in a dose-dependent manner over a seven day period of BMP-7 treatment, with a maximum increase of approximately 5-fold at the higher dose of BMP-7. In contrast, GDF-5 mRNA expression decreased in response to BMP-7 treatment by approximately 80% relative to controls. These data were statistically significant at both 40 and 400 ng/ml BMP-7 (p<0.05).

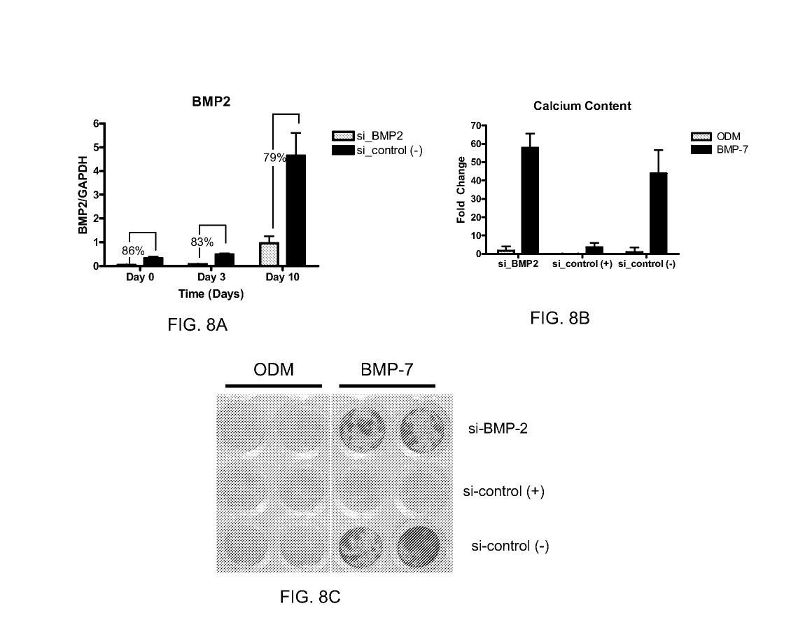

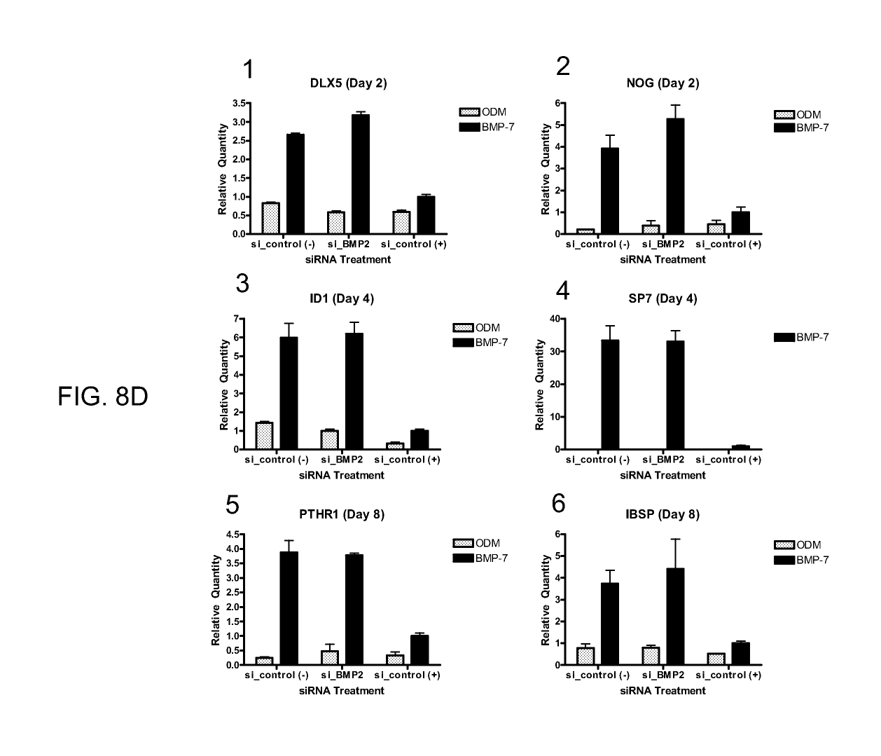

We next investigated whether the osteoinductive activities of BMP-7 are exerted independently or in synergy with endogenous BMP-2. BMP-7 mediated osteoblastic differentiation was evaluated in hMSC in which a potent and sustained down-regulation of BMP-2 was achieved using siRNA. Primary hMSC were nucleoporated with a total of 4 μg siRNA targeting BMP-2 or positive or negative control siRNAs. 48 hours after nucleoporation, cells were treated with ODM alone or ODM containing 200 ng/ml BMP-7. Cells were harvested at Day 0, 3 or 10 after BMP-7 treatment (2, 5 and 12 days after nucleofection, respectively) to assess target gene knockdown by RT-QPCR, with data showing in FIG. 8A. Cells were stained with Alizarin red after 12 days of BMP-7 treatment, to assess mineralization as shown in FIG. 8B. Calcium content was assessed after 10 days of BMP-7 treatment as shown in FIG. 8C.

Alizarin Red staining revealed that siRNA mediated knockdown of BMP-2 did not block the ability of BMP-7 treated hMSC to mineralize. In contrast, no mineralization was detected in cells nucleoporated with pooled siRNA targeting BMPR1A and ACVR1A (FIG. 8B). These data were confirmed through quantification of calcium content (FIG. 8C), and supported by an assessment of osteoblast associated gene expression by QPCR (FIG. 8D).

A range of relevant phenotypic marker genes was evaluated, including DLX5 and NOG at day 2, ID1 and SP7 at day 4, and PTHR1 and IBSP at day 8. Cells were lysed after 2 days of BMP-7 treatment to assess DLX5 and Noggin gene expression, after four days to assess ID1 and SP7 gene expression, and after 8 days to asses PTHR1 and IBSP gene expression. Levels of gene expression were quantified by RT-QPCR and normalized to GAPDH. All genes were potently induced by BMP-7 in the negative control siRNA treatments but not after nucleofection with the positive control siRNAs (p<0.05). No significant effect of BMP-2 knockdown was observed relative to the negative control siRNA for any of the six phenotypic marker genes. These data indicate that endogenous BMP-2 expression is not required for in vitro BMP-7 mediated induction of matrix mineralization and osteoblast associated gene expression, and confirm that the gene modulation reported in this study is attributable to the direct osteoinductive bioactivity of BMP-7 alone.

### Example 9

### The Effect of BMP-7 on Expression of BMP Inhibitors

We further interrogated the unfiltered RMA normalized dataset, as described above and elsewhere herein, to evaluate the expression of BMP inhibitors in BMP-7 treated hMSC.

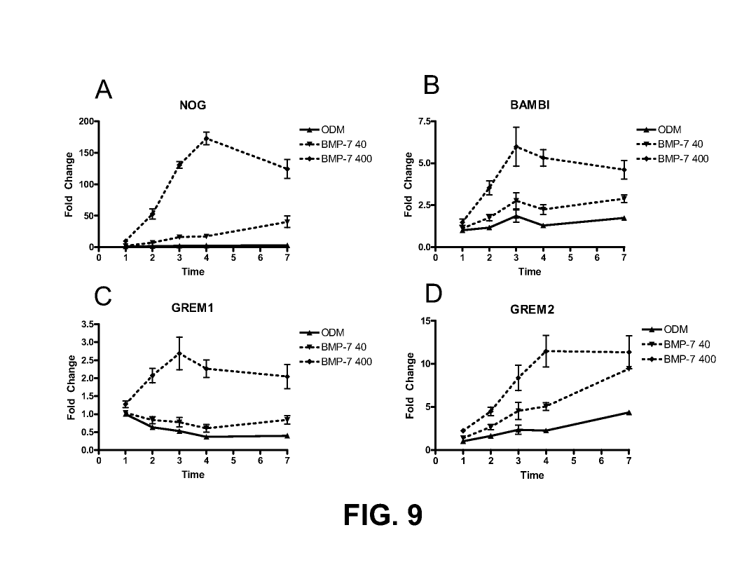

Primary hMSC were seeded in 48-well dishes at 1.5×10⁴ cells per well and cultured in ODM alone or ODM supplemented with 40 or 400 ng/ml BMP-7. Cells were lysed after 1, 2, 3, 4 or 7 days of treatment. A subset of genes demonstrating a statistically significant modulation of ≧20% in BMP-7 versus control treated cells were further investigated by QPCR. Of the genes evaluated, most were not found to be regulated by BMP-7 in follow-up studies. Expression of NOG, BAMBI, GREM1, and GREM2 was quantified by RT-QPCR and normalized to GAPDH. Treatment at both 40 and 400 ng/ml of BMP-7 led to a significant (p<0.05) up-regulation of NOG, BAMBI, GREM1 and GREM2 (FIG. 9), verifying the trends suggested by the Affymetrix data and confirming BMP-responsiveness of these genes as demonstrated previously (Gazzerro et al., (1998), J. Clin. Invest., 102:2106-14; Diefenderfer et al., (2003), J Bone Joint Surg Am, 85-A Supp 13:19-28; Pereira et al., (2000), Endocrinology, 141:4558-63; Groewold et al., (2001), Mech Dev, 100:327-30). As suggested by the Affymetrix data, a significant (p<0.05) up-regulation of SOST was observed at the highest dose of BMP-7, although the overall level of expression of SOST remained extremely low (data not shown). The physiological relevance of this low level of induction is unclear. Noggin expression was notable for the magnitude of induction, with expression levels reaching approximately 170-fold over controls at the higher dose of BMP-7.

### Example 10

### Promoting Endochondral Bone Formation in a Patient

A patient presents with a non-union bone fracture that has not healed naturally for three months. A population of mesenchymal stem cells is prepared for administration to the patient at the site of the fracture. The mesenchymal stem cells are contacted with 1000 μg

BMP-7 and an effective concentration of an FGFR3-specific FGF on day 1, day 2, day 4 and day 7. At day 7, the mesenchymal cell population is provided to the patient via injection to the site of the bone fracture. The cell population is provided in an osteoinductive or an osteoconductive matrix. The fracture is monitored by X-ray to determine presence of endochondral bone growth. Within 1 month from administration of the mesenchymal stem cells, X-rays show that the fracture is healed.

1. A method of promoting endochondral bone formation, the method comprising the step of:
inhibiting or down-regulating the activity of deiodinase and/or the DIO2 gene.

2. The method of claim 1, wherein inhibiting or down-regulating the activity of deiodinase and/or the DIO2 gene comprises administering an antagonist of deiodinase and/or an inhibitor of DIO2 gene expression.

3. A method of promoting endochondral bone formation, the method comprising the step of:
inducing or up-regulating the activity of the gene, or its expression product, selected from the group consisting of: FGFR3, ADAMTS9, HEY1, HAS3 and MFI2.

4. The method of claim 3, wherein inducing or up-regulating the activity of the gene comprises administering BMP-7.

5. The method of claim 3, wherein inducing or up-regulating the activity of the expression product comprises administering an agonist of the expression product.

6. The method of claim 5, wherein the gene is FGFR3 and the agonist is an FGF specific for binding to FGFR3.

7. A composition for promoting endochondral bone formation comprising:
BMP-7; and
an agonist of one or more of the FGFR3, ADAMTS9, HEY1, HAS3 and MFI2 proteins.

8. The composition of claim 7, further comprising mesenchymal stem cells.

9. A method of promoting endochondral bone formation, the method comprising the steps of:
(a) administering an effective amount of an agent which reduces or blocks the activity of deiodinase and/or the DIO2 gene in combination with an effective amount of BMP-7 and/or BMP-7 agonist, wherein the administering step induces osteoblastogenesis but not mineralization of osteoblasts resulting therefrom;
(b) allowing accumulation of non-mineralized osteoblasts; and,
(c) reversing or neutralizing the agent's block of deiodinase and/or the DIO2 gene,
wherein accumulated osteoblasts are mineralized and endochondral bone formation occurs.

10. The method of claim 9, wherein the reversing or neutralizing step is accomplished upon metabolic depletion or exhaustion of the agent over time.

11. The method of claim 9, wherein the reversing or neutralizing step is accomplished by providing: T3; an agonist of deiodinase; and/or an agonist of the DIO2 gene.

12. The method of claim 9, wherein the effective amount of BMP-7 is endogenous BMP-7.

13. The method of claim 9, further comprising the step of administering in step (a) or (b) an effective amount of an agent which reduces or blocks the activity of Noggin and/or the Noggin gene.

14-18. (canceled)

19. A composition comprising:
stem cells;
BMP-7 or mimetic or agonist thereof; and,
an antagonist of deiodinase and/or the DIO2 gene and/or T3.

20. The composition of claim 19, further comprising an antagonist of Noggin and/or the Noggin gene.

21. (canceled)

22. A method of modulating endochondral bone formation, the method comprising the step of:
providing BMP-7 or mimetic or agonist thereof in an amount effective to down-regulate CHI3L1 gene activity,
wherein bone deposition follows CHI3L1 down-regulation.

23. A method of tissue engineering, the method comprising the step of:
providing BMP-7 or mimetic or agonist thereof in an amount effective continuously to down-regulate osteoclastic events comprising modulation of chemokine- or cytokine-induced osteoclastogenesis.

24. A method of preparing non-mineralized differentiated osteoblasts, the method comprising the step of:
(a) contacting human mesenchymal stem cells with an effective amount of BMP-7 and an effective amount of an agent which reduces or blocks the activity of deiodinase and/or the DIO2 gene,
wherein the cells undergo osteoblastogenesis to form differentiated osteoblasts which are non-mineralized.

25-26. (canceled)

27. A method of promoting osteoblastogenesis of non-mineralized osteoblasts, the method comprising the step of:
(a) contacting human mesenchymal stem cells with an effective amount of BMP-7 and an effective amount of an agent which reduces or blocks the activity of deiodinase and/or the DIO2 gene,
wherein the cells undergo osteoblastogenesis to form differentiated osteoblasts which are non-mineralized.

28. A method of arresting osteoblastic differentiation of human mesenchymal stem cells, the method comprising the step of:
(a) contacting human mesenchymal stem cells with an effective amount of BMP-7 and an effective amount of an agent which reduces or blocks the activity of deiodinase and/or the DIO2 gene,
wherein osteoblastic differentiation is arrested such that the cells undergo osteoblastogenesis but not mineralization.

29. A method of continuous cultivation of partially differentiated human mesenchymal stem cells, the method comprising the step of:
(a) contacting human mesenchymal stem cells with an effective amount of BMP-7 and an effective amount of an agent which reduces or blocks the activity of deiodinase and/or the DIO2 gene,
wherein the cells undergo osteoblastogenesis to form partially differentiated cells.
